(19) United States
(12) Patent Application Publication
(10) Pub. No.: US 2017/0095496 A1
(43) Pub. Date: Apr. 6, 2017

(54) GALACTOSIDE INHIBITORS FOR NEW USES

(71) Applicant: GALECTO BIOTECH AB, COPENHAGEN (DK)

(72) Inventors: Tomas DEIERBORG, Loddekopinge (SE); Antonio SERRANO, Lund (SE); Hakon LEFFLER, Lund (SE); Ulf NILSSON, Lund (SE)

(73) Assignee: GALECTO BIOTECH AB, COPENHAGEN (DK)

(21) Appl. No.: 15/300,769

(22) PCT Filed: Apr. 8, 2015
(86) PCT No.: PCT/EP2015/057565

§ 371 Date: Sep. 29, 2016

(30) Foreign Application Priority Data
Apr. 8, 2014 (EP) 14163872.6

(51) Int. Cl. A61K 31/7056; A61K 9/00

(52) U.S. Cl. CPC A61K 31/7056; A61K 9/0085

(57) ABSTRACT

Provided is a method for treatment or prevention of α-synucleinopathies in a mammalian subject, the method comprising administering a therapeutically effective amount of at least one composition to the subject, wherein the composition comprises a molecule for pharmacological modulation of galectin activity in a mammalian brain.

### TECHNICAL FIELD

The present invention relates to a composition for use in a method for treatment or prevention of α-synucleinopathies wherein the composition comprises a molecule for pharmacological modulation of galectin activity in a mammalian brain. The invention also relates to pharmaceutical compositions comprising said molecules. Furthermore, the present invention relates to a method for treatment or prevention of α-synucleinopathies in a mammalian subject.

### BACKGROUND ART

Neuroinflammation and microglial cells are involved in several acute and chronic diseases to the central nervous system (CNS). The control of microglial activation is a relevant therapeutic target for both slow progressing neurodegenerative diseases as for acute injuries to CNS (Lyman et al., 2014). α-synuclein is a synaptic protein that is believed to be implicated in several neurodegenerative disorders including the typical α-synucleinopathies (Parkinson Disease (PD), dementia with Lewy bodies and multiple system atrophy. A general feature of all neurodegenerative conditions is an activation of microglial cells.

Parkinson's Disease (PD) is a progressive motor neurodegenerative disorder characterized by bradykinesia, rigidity and tremor and affects about 1% of the population over 60 years of age (Samii et al., 2004, Casey, 2013). Pathologically, PD is characterized by glial activation, brain inflammation, progressive dopaminergic cells degeneration (Qiao et al., 2012) and the α-synuclein accumulation into hallmark protein inclusions termed Lewy bodies and Lewy neurites (Spillantini et al., 1997). It is thought that microglial activation during the inflammatory response is implicated in neuronal degeneration in PD (Tomas-Camardiel et al., 2004, Villar-Cheda et al., 2012). However, the process that modulates microglial response and the neurodegeneration is still yet to be elucidated. A clear link between PD and α-synuclein has been shown by mutations in α-synuclein gene (SNCA) which can participate in the pathogenis of PD (Kruger et al., 1998). α-synuclein proteins are found mainly in presynaptic terminals, it is very abundant in human brain, and it is related with: synaptic rearrangement, neuronal development and neuronal plasticity (George et al., 1995, Kholodilov et al., 1999, Stefanis, 2012) among other features. It is mainly expressed in neurons but also expressed in different cell types including the immune cells (T-cells, B-cells and natural killer cells) as well as monocytes. Furthermore, secreted α-synuclein may exert deleterious effects on neighboring cells, including seeding of aggregation, thus possibly contributing to disease propagation (Stefanis, 2012, Lee et al., 2014).

The role of α-synuclein in the inflammatory microglial response has been demonstrated both in vitro and in vivo. For instance, α-synuclein treatment can trigger IL-1β release in monocytes (Codolo et al., 2013) and Toll-like receptor 2 (TLR2) (Kim et al., 2013) and Toll-Like Receptor 4 (TLR4) (Fellner et al., 2013) activation has been identified as receptors involved in α-synuclein-induced activation of microglia. To date, different laboratories have reported the effects of extracellular α-synuclein on microglia activation and the inflammatory response. For instance, Codolo et al have reported that fibrillar α-synuclein can trigger an inflammatory response. Kim et al. have reported that specifically neuron-released oligomeric α-synuclein can also trigger microglial activation. These findings demonstrate that the effects on microglial response depends on the origin of the protein (cell derived vs recombinant), the type of protein used (WT or mutant) (Rojanathammanee et al., 2011), or the molecular state in which the protein is produced (monomeric, oligomeric or fibrillar) may have different effects on the microglia.

Activated microglia can develop mainly two well-characterized profiles namely as alternative (anti-inflammatory) and classical profile (pro-inflammatory) in which different surface proteins allow microglial cells to sense the environment. Depending on the stimulus, microglial cells can shift from a resting state to an inflammatory or anti-inflammatory profile. In the pro-inflammatory profile, microglial cells release different pro-inflammatory molecules (TNF-α, IL-1β, IL-12, IFN-γ or Nitric Oxide) that have been shown to decrease neuronal survival (De Pablos et al., 2005, Zindler and Zipp, 2010). The alternative profile is characterized by the release of anti-inflammatory factors (IL-4, IL-13, TGF-β) that can reduce microglial activation (Stirling et al., 2013). Different pathways have been suggested to be implicated in α-synuclein microglial activation including ERK 1/2, p38 MAPK, inflammasome pathway and NF-κβ, but the role of inflammatory modulators, for example galectin-3, has not been studied. Galectin-3 is expressed in a wide range of cells, including immune cells including microglial upon activation and is a molecule implicated in disorders such as encephalomyelitis, traumatic brain injury, EAE and ischemic brain injury (Jiang et al., 2009, Lalancette-Hebert et al., 2012, Pajoohesh-Ganji et al., 2012). However, the role of galectin-3 in pathological processes related to PD has not been studied yet.

Galectin-3 is a member of the β-galactoside-binding lectin family and it is composed of a carbohydrate recognition domains (CRD) linked to a non-CRD N-terminus. Previous studies have shown that galectin-3 plays a role in different biological activities, including: cell adhesion, proliferation, clearance (Lee et al., 2008), apoptosis, cell activation, cell migration (Shin, 2013) and inflammatory regulation (Liu and Rabinovich, 2010). Galectin-3 can be found extracellular and intracellular in different tissues, and in the cytoplasm or the nucleus as well (Yang et al., 1996). Current evidence suggests that galectin-3 plays a role in both, pro-inflammatory and anti-inflammatory profile depending on cell type and the kind of insult (Shin, 2013).

### SUMMARY OF THE DISCLOSURE

The present inventors have demonstrated that α-synuclein, particularly in its aggregated form, induces microglial activation. We show that galectin-3 plays a significant role in the pro-inflammatory response of microglia induced by α-synuclein. In particular galectin-3 has been shown to be rate limiting for the inflammatory response caused by alfa-synuclein in microglia. A significant increase of the inflammatory activation measured as an increase of several proinflammatory markers such as: inducible Nitric Oxide Synthetase (iNOS), phagocytosis and proinflammatory cytokines (IL-1β and IL-12) in the murine microglial cell line BV2 upon α-synuclein treatment was found. By decreasing galectin-3 levels using small interfering RNA (siRNA), using microglia from galectin-3 KO mice or pharmacological intervention of galectin-3 activity using bis-{3-deoxy-3-[4-(3-fluorophenyl)-1H-1,2,3-triazol-1-yl]-β-D-galactopyranosyl} sulfane (a known galectin-3 inhibitor), a significant reduction of the inflammatory response induced by α-synuclein was found.

Taken together, the current data suggest that pharmacological modulation of galectin activity, in particular galectin-3 activity, might constitute a novel therapeutic approach for neurodegenerative diseases including α-synucleinopathies (such as Parkinson's Disease (PD), dementia with Lewy bodies and multiple system atrophy) since it decreases α-synuclein-induced inflammatory response and therefore could help to prevent the neuronal loss.

α-Synuclein is suggested to be implicated in Alzheimer's disease, where soluble α-synuclein is increased twofold in patients with Alzheimer's disease and correlate stronger to cognitive impairment than the typical Alzheimer's disease related proteins Aβ and tau levels (Larson et al., 2012).

Multiple Sclerosis is a neurodegenerative disease with a strong inflammatory component where α-synuclein is expressed in microglia, at lesion sites, and can contribute to the disease progression (Lu et al., 2009). α-synuclein is also implicated in ischemic stroke, where α-synuclein fibrillization in the ischemic brain lead to formation of α-synuclein aggregates in normal mice and where ischemia in mice overexpressing α-synuclein (and thereby increased formation of α-synuclein fibrils) have larger infarcts.

Epilepsia is associated with activation of infarcts (Unal-Cevik et al., 2011)). Activated microglial cells can be found at the epileptogenic site in the brain where the α-synuclein protein level is increased ((Li et al., 2010)), thereby providing α-synuclein the opportunity to induce microglia activation and contributing to disease progression.

CNS trauma increases α-synuclein levels in the cerebral spinal fluid, which is suggested to reflect secondary events of the neuropathology ((Mondello et al., 2013)), potentially including activation of microglial cells.

Infantile neuroaxonal dystrophy (INAD) is a neurodegenerative disorder characterized by α-synuclein protein aggregation ((Riku et al., 2013)) and microglial activation ((Zhao et al., 2011)) and thereby a potential implication of α-synuclein-induced microglia activation.

Alzheimer's disease (AD) is the most common neurodegenerative disease, and it often coexists with vascular dementia. Epidemiological and clinical studies have shown that neuroinflammation plays a key role in the disease process (Heneka et al., 2015). Central to AD pathogenesis is the formation of plaques in the brain. These plaques are made of aggregated amyloid-β (Aβ), and it is the accumulation of these aggregates that drives neuroinflammation. Microglia, the primary immune cell of the brain, surround Aβ plaques in an attempt to clear them by phagocytosis and degradation. These cells have evolutionarily evolved to react to specific molecules, allowing for a quick response against external agents such as bacteria. Aβ in the brain is almost identical to the molecular structures present on bacteria (Wang et al., 2008). In the brain, an uncontrolled or chronic inflammatory response can damage nerve cells.

In reaction to an acute inflammatory event, there are several regulatory mechanisms that will dampen the inflammation in the brain in response to the peripheral inflammation (Heneka et al., 2015). However, the neuroinflammation in Alzheimer's disease is a chronic inflammation, because microglia have already been primed and are therefore responsive to further activation, causing a rapid switch to a detrimental M1 microglia phenotype (Heneka et al., 2015).

The carbohydrate-binding protein gal3 is suggested to be important in several inflammatory conditions including CNS diseases and chronic inflammation (Henderson and Sethi, 2009). We have recently identified a new molecular pathway in innate immunity; gal3 produced by microglia can bind TLR4, a key receptor in inflammation (Burguillos et al., 2015). In this study we have shown that lack of gal3 is neuroprotective in animal models of Stroke/brain ischemia and in models for Parkinson's disease (Burguillos et al., 2015).

In a first aspect the present invention concerns a composition, such as a pharmaceutical composition, for use in a method for treatment or prevention of α-synucleinopathies wherein the composition comprises a molecule for pharmacological modulation of galectin activity, typically, a molecule for pharmacological modulation of galectin activity in a mammalian brain, such as a human brain.

In one embodiment of the composition the galectin activity is galectin-3 activity.

In a further embodiment the pharmacological modulation of galectin activity is inhibition of galectin activity, such as galectin-3 activity.

In a still further embodiment the molecule is selected from at least one of: a drug, a polymer, a protein, a peptide, a carbohydrate, a low molecular weight compound, an oligonucleotide, a polynucleotide, and a genetic material such as DNA or RNA. Typically, the molecule is a beta-galactoside, which is derivatized or functionalized. In a specific embodiment the molecule is selected from a low molecular weight compound comprising a carbohydrate selected from a glycopyranose. In another specific embodiment the molecule is selected from a low molecular weight compound comprising a carbohydrate selected from a thio-digalactoside. In another specific embodiment the molecule is selected from a low molecular weight compound comprising a carbohydrate selected from a C3-[1,2,3]-triazol-1-yl-D-galactose. In another specific embodiment the molecule is selected from a low molecular weight compound comprising a carbohydrate selected from a C3-[1,2,3]-triazol-1-yl-1-thio-D-galactose. Preferably the low molecular weight compound is below 1000 Da, such as below 500 Da.

In a further embodiment the composition is effective in a method to treat or prevent a disease or a condition associated with α-synucleinpathies with inflammatory features.

In a still further embodiment the disease or condition is selected from a neurodegenerative disease or condition. Further embodiments of the neurodegenerative disease or condition is selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy. In a particular embodiment the α-synucleinopathies is selected from Parkinson's disease. In another embodiment the α-synucleinopathies is selected from dementia with Lewy bodies. In another embodiment the α-synucleinopathies is selected from pure autonomic failure (PAF). In another embodiment the α-synucleinopathies is selected from Alzheimer's disease. In another embodiment the α-synucleinopathies is selected from neurodegeneration with brain iron accumulation. In another embodiment the α-synucleinopathies is selected from adult neuroaxonal dystrophy or Hallervorden-Spatz syndrome. In another embodiment the α-synucleinopathies is selected from traumatic brain injury. In another embodiment the α-synucleinopathies is selected from amyotrophic lateral sclerosis. In another embodiment the α-synucleinopathies is selected from Pick disease. In another embodiment the α-synucleinopathies is selected from multiple system atrophy (including Shy-Drager syndrome, striatonigral degeneration, and olivopontocerebellar atrophy). In another embodiment the α-synucleinopathies is selected from stroke. In another embodiment the α-synucleinopathies is selected from multiple sclerosis. In another embodiment the α-synucleinopathies is selected from epilepsy. In another embodiment the α-synucleinopathies is selected from infantile neuroaxonal dystrophy.

In a further embodiment the molecule has the following general formula:

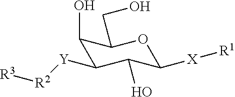

wherein the configuration of the pyranose ring is D-galacto;

X is selected from the group consisting of O, S, NH, CH₂, and NR⁴, or is a bond;

Y is selected from the group consisting of NH, CH₂, and NR⁴, or is a bond;

R¹ is selected from the group consisting of: a saccharide; hydrogen, an alkyl group, an alkenyl group, an aryl group, a heteroaryl group, and a heterocycle;

R² is selected from the group consisting of CO, SO₂, SO, PO, and PO₂;

R³ is selected from the group consisting of: an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, and an alkyl group of at least 4 carbon atoms substituted with a halogen; a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxy group, a phenyl group substituted with a carbonyl group and a phenyl group substituted with a substituted carbonyl group; and a phenyl amino group;

R⁴ is selected from the group consisting of hydrogen, an alkyl group, an alkenyl group, an aryl group, a heteroaryl group, and a heterocycle. In one embodiment R¹ is a saccharide selected from the group consisting of glucose, mannose, galactose, N-acetylglucosamine, N-acetylgalactosamine, fucose, fructose, xylose, sialic acid, glucuronic acid, iduronic acid, a disaccharide or, an oligosaccharide comprising at least two of the above saccharides, and derivatives thereof.

In a further embodiment Y is NH.

In a still further embodiment X is O.

In a further embodiment the halogen is individually selected from the group consisting of F, Cl, Br and I.

In a still further embodiment the molecule is selected from methyl 2-acetamido-2-deoxy-4-O-(3-[3-carboxypropanamido]-3-deoxy-β-D-galactopyranosyl)-β-D-glucopyranoside; methyl 2-acetamido-2-deoxy-4-O-(3-[{Z}-3-carboxypropenamido]-3-deoxy-β-D-galactopyranosyl)-β-D-glucopyranoside; methyl 2-acetamido-2-deoxy-4-O-(3-benzamido-3-deoxy-β-D-galactopyranosyl)-β-D-glucopyranoside; methyl 2-acetamido-2-deoxy-4-O-(3-[2-carboxy-benzamido]-3-deoxy-β-D-galactopyranosyl)-β-D-glucopyranoside; methyl 2-acetamido-2-deoxy-4-O-(3-[4-methoxy-2,3,5,6-tetrafluorbenz-amido]-3-deoxy-β-D-galactopyranosyl)-β-D-glucopyranoside; methyl 2-acetamido-2-deoxy-4-O-(3-[2-carboxy-3,4,5,6-tetrafluorbenzamido]-3-deoxy-β-D-galactopyranosyl)-β-D-glucopyranoside; methyl 2-acetamido-2-deoxy-4-O-(3-methanesulfonamido-3-deoxy-β-D-galactopyranosyl)-β-D-glucopyranoside; methyl 2-acetamido-2-deoxy-4-O-(3-[-4-nitrobenzenesulfonamido]-3-deoxy-β-D-galactopyranosyl)-β-D-glucopyranoside, methyl 2-acetamido-2-deoxy-4-O-(3-phenylaminocarbonylam-ino-3-deoxy-β-D-galactopyranosyl)-β-D-glucopyranoside; methyl 2-acetamido-2-deoxy-4-O-(3-aminoacetamido-3-deoxy-β-D-galactopyranosyl)-β-D-glucopyranoside; and methyl 2-acetamido-2-deoxy-4-O-(-3-[{2S}-2-amino-3-carboxy-propanamido]-3-deoxy-β-D-galactopyranosyl)-β-D-glucopyranoside.

In another embodiment the molecule has the general formula:

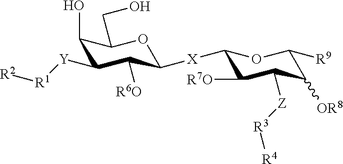

wherein the configuration of one of the pyranose rings is β-D-galacto;

X is selected from the group consisting of O, S, SO, SO₂, NH, CH₂, and NR⁵,

Y is selected from the group consisting of O, S, NH, CH₂, and NR⁵, or is a bond;

Z is selected from the group consisting of O, S, NH, CH₂, and NR⁵, or is a bond;

R¹ and R³ are independently selected from the group consisting of CO, SO₂, SO, PO₂, PO, and CH₂ or is a bond;

R² and R⁴ are independently selected from the group consisting of: an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamnino group, a phenyl group substituted with at least one hydroxy group, a phenyl group substituted with at least one carbonyl group and a phenyl group substituted with at least one substituted carbonyl group; or a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamnino group, a naphthyl group substituted with at least one hydroxy group, a naphthyl group substituted with at least one carbonyl group and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group; R⁶ and R⁸ are independently selected from the group consisting of a hydrogen, an acyl group, an alkyl group, a benzyl group, and a saccharide; R⁷ is selected from the group consisting of a hydrogen, an acyl group, an alkyl group, and a benzyl group; R⁹ is selected from the group consisting of a hydrogen, a methyl group, hydroxymethyl group, an acyloxymethyl group, an alkoxymethyl group, and a benzyloxymethyl group.

In one embodiment Y is NH. In a further embodiment Z is NH. In a still further embodiment X is S. In a further embodiment R¹ is CO. In a still further embodiment R³ is CO. In a further embodiment R² or R⁴ is an aromatic for example an aromatic ring; either of R⁶, R⁷, and R⁸ is hydrogen; or R⁹ is a hydroxymethyl group.

In a still further embodiment the molecule is selected from bis-(3-deoxy-3-benzamido-β-D-galactopyranosyl)sulfane, bis-(3-deoxy-3-(3-methoxybenzamido)-β-D-galactopyranosyl)sulfane; bis-(3-deoxy-(3,5-dimethoxybenzamido)-β-D-galactopyranosyl)sulfane; bis-(3-deoxy-3-(4-nitrobenzamido)-β-D-galactopyranosyl)sulfane; bis(3-deoxy-3-(2-naphthamido)-β-D-galactopyranosyl)sulfane; bis-(3-deoxy-3-(4-methoxybenzamido)-β-D-galactopyranosyl)sulfane; bis-(3-deoxy-3-(3-nitrobenzamido)-β-D-galactopyranosyl)sulfane; bis-(3-deoxy-3-[4-(dimethylamino)-benzamido]-β-D-galactopyranosyl)sulfane; bis-(3-deoxy-3-(4-methylbenzamido)-β-D-galactopyranosyl)sulfane; bis-(3-deoxy-3-(4-chlorobenzamido)-β-D-galactopyranosyl)sulfane; bis-(3-deoxy-3-(4-tert-butylbenzamido)-β-D-galactopyranosyl)sulfane; bis-(3-deoxy-3-(4-acetylbenzamido)-β-D-galactopyranosyl)sulfane; bis-(3-deoxy-3-[2-(3-carboxy)-naphthamido]-β-D-galactopyranosyl)sulfane; bis-[3-deoxy-3-(3,4-methylenedioxy)benzamido]-β-D-galactopyranosyl)sulfane, bis-(3-deoxy-3-(4-methoxycarbonylbenzamido)-β-D-galactopyranosyl)sulfane; bis-(3-deoxy-3-(3-carboxybenzamido)-β-D-galactopyranosyl)sulfane; bis-(3-deoxy-3-(3-benzyloxy-5-hydroxy-benzamido)-β-D-galactopyranosyl)sulfane; bis-(3-deoxy-3-(3,5-dibenzyloxybenzamido)-β-D-galactopyranosyl)sulfane; bis-(3-deoxy-3-(3-benzyloxy-5-methoxy-benzamido)-β-D-galactopyranosyl)sulfane; bis-(3-deoxy-3-(3-benzyloxy-5-nonyloxy-benzamido)-β-D-galactopyranosyl)sulfane; bis-(3-deoxy-3-(3-hydroxy-5-methoxy-benzamido)-β-D-galactopyranosyl)-sulfane; bis-(3-deoxy-3-(3-hydroxy-5-nonyloxy-benzamido)-β-D-galactopyranosyl)sulfane, bis-(3-deoxy-3-[3-benzyloxy-5-(4-fluoro-benzyloxy)-benzamido]-β-D-galactopyranosyl)sulfane; bis-(3-deoxy-3-[3-methoxy-5-(4-methyl-benzyloxy)-benzamido]-β-D-galactopyranosyl)sulfane; and bis-(3-deoxy-3-(3-allyloxy-5-benzyloxy-benzamido)-β-D-galactopyranosyl)sulfane.

In another embodiment the molecule has the general formula:

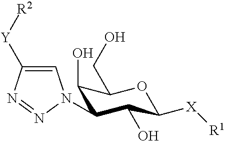

wherein the configuration of the pyranose ring is D-galacto;

X is selected from the group consisting of O, S, NH, CH₂, and NR⁴, or is a bond;

Y is selected from the group consisting of CH₂, CO, SO₂, SO, PO₂ and PO, phenyl, or is a bond;

R₁ is selected from the group consisting of: a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives thereof; and an amino group, a substituted amino group, an imino group, or a substituted imino group; and

R² is selected from the group consisting of hydrogen, an amino group, a substituted amino group, an alkyl group, a substituted alkyl group, an alkenyl group, a substituted alkenyl group, an alkynyl group, a substituted alkynyl group, an alkoxy group, a substituted alkoxy group, an alkylamino group, a substituted alkylamino group, an arylamino group, a substituted arylamino group, an aryloxy group, a substituted aryloxy group, an aryl group, a substituted aryl group, a heteroaryl group, a substituted heteroaryl group, and a heterocycle, a substituted heterocycle.

In one embodiment R¹ is a saccharide selected from the group consisting of glucose, mannose, galactose, N-acetylglucosamine, N-acetylgalactosamine, fucose, fructose, xylose, sialic acid, glucuronic acid, iduronic acid, galacturonic acid, a disaccharide or an oligosaccharide comprising at least two of the above saccharides, and derivatives thereof. Typically, R¹ is galactose, glucose or N-acetylglucosamine. In another embodiment R¹ is a substituted galactose. In a further embodiment R¹ is either a substituted glucose, or a substituted N-acetylglucosamine. In a another embodiment R¹ is a C3-[1,2,3]-triazol-1-yl-substituted galactose.

In a further embodiment Y is CO, SO₂, or a bond.

In a still further embodiment R² is an amine or an aryl group, or R² is a substituted phenyl group wherein said substituent is one or more selected from the group consisting of halogen, alkoxy, alkyl, nitro, sulfo, amino, hydroxy or carbonyl group.

In a further embodiment X is O or S.

In a still further embodiment the molecule is selected from methyl 3-deoxy-3-(1H-[1,2,3]-triazol-1-yl)-1-thio-β-D-galactopyranoside; methyl 3-deoxy-3-(4-propyl-1H-[1,2,3]-triazol-1-yl)-1-thio-β-D-galactopyranoside; methyl 3-(4-methoxycarbonyl-1H-[1,2,3]-triazol-1-yl)-3-deoxy-1-thio-β-D-galactopyranoside; methyl 3-deoxy-3-(4-(1-hydroxy-1-cyclohexyl)-1H-[1,2,3]-triazol-1-yl)-1-thio-β-D-galactopyranoside; methyl 3-deoxy-3-(4-phenyl-1H-[1,2,3]-triazol-1-yl)-1-thio-β-D-galactopyranoside; methyl 3-deoxy-3-(4-p-tolylsulfonyl-1H-[1,2,3]-triazol-1-yl)-1-thio-β-D-gal-actopyranoside; methyl 3-(4-methylaminocarbonyl-1H-[1,2,3]-triazol-1-yl)-3-deoxy-1-thio-β-D-galactopyranoside; methyl 3-(4-butylaminocarbonyl-1H-[1,2,3]-triazol-1-yl)-3-deoxy-1-thio-β-D-galactopyranoside; methyl 3-(4-benzylaminocarbonyl-1H-[1,2,3]-triazol-1-yl)-3-deoxy-1-thio-β-D-galactopyranoside; methyl 3-{4-(3-hydroxyprop-1-ylaminocarbonyl)-1H-[1,2,3]-triazol-1-yl}-3-deoxy-1-thio-β-D-galactopyranoside; methyl 3-{4-[2-(N-morpholino)-ethylaminocarbonyl]-1H-[1,2,3]-triazol-1-yl}-3-deoxy-1-thio-β-D-galactopyranoside; methyl 3-(4-methylaminocarbonyl-1H-[1,2,3]-triazol-1-yl)-3-deoxy-β-D-galactopyranosyl-(1→4)-2-acetamido-2-deoxy-β-D-glucopyranoside, bis-{3-deoxy-3-[4-(3-fluorophenyl)-1H-1,2,3-triazol-1-yl]-β-D-galactopyranosyl} sulfane, methyl 3-deoxy-3-{4-(2-fluorophenyl)-1H-[1,2,3]-triazol-1-yl}-1-thio-β-D-galactopyranoside; methyl 3-deoxy-3-{4-(2-methoxyphenyl)-1H-[1,2,3]-triazol-1-yl}-1-thio-β-D-galactopyranoside; methyl 3-deoxy-3-{4-(3-methoxyphenyl)-1H-[1,2,3]-triazol-1-yl}-1-thio-β-D-galactopyranoside; methyl 3 deoxy-3-{4-(4-methoxyphenyl)-H-[1,2,3]-triazol-1-yl}-1-thio-β-D-galactopyranoside; methyl 3-deoxy-3-{4-(3,5-dimethoxyphenyl)-1H-[1,2,3]-triazol-1-yl}-1-thio-β-D-galactopyranoside; methyl 3-deoxy-3-{4-(1-naphthyl)-1H-[1,2,3]-triazol-1-yl}-1-thio-β-D-galactopyranoside; methyl 3-deoxy-3-{4-(2-naphthyl)-1H-[1,2,3]-triazol-1-yl}-1-thio-β-D-galactopyranoside; methyl 3-deoxy-3-{4-(2-pyridyl)-1H-[1,2,3]-triazol-1-yl}-1-thio-β-D-galactopyranoside; methyl 3-deoxy-3-{4-(3-pyridyl)-1H-[1,2,3]-triazol-1-yl}-1-thio-β-D-galactopyranoside; methyl 3-deoxy-3-{4-(4-pyridyl)-1H-[1,2,3]-triazol-1-yl}-1-thio-β-D-galactopyranoside; O-{3-deoxy-3-[4-phenyl-[1H-[1,2,3]-triazol-1-yl]-β-D-galactopyranosy-1}-3-indol-carbaldoxim; O-{3-deoxy-3-[4-(methylaminocarbonyl)-1H-[1,2,3]-triazol-1-yl]-β-D-galactopyranosyl}-3-indol-carbaldoxim; O-{3-deoxy-3-[4-phenyl-[1H-[1,2,3]-triazol-1-yl]-β-D-galactopyranosy-1}-(2-hydroxy-5-nitro-phenyl)-carbaldoxim; O-{3-deoxy-3-[4-(methylaminocarbonyl)-1H-[1,2,3]-triazol-1-yl]-β-D-galactopyranosyl}-(2-hydroxy-5-nitro-phenyl)-carbaldoxim; O-{3-deoxy-3-[4-phenyl-[1H-[1,2,3]-triazol-1-yl]-β-D-galactopyranosy-1}-(2,5-dihydroxyphenyl)-carbaldoxim; O-{3-deoxy-3-[4-(methylaminocarbonyl)-1H-[1,2,3]-triazol-1-yl]-β-D-galactopyranosyl}-(2,5-dihydroxyphenyl)-carbaldoxim; O-{3-deoxy-3-[4-phenyl-[1H-[1,2,3]-triazol-1-yl]-β-D-galactopyranosy-1}-1-naphthyl-carbaldoxim; and O-{3-deoxy-3-[4-(methylaminocarbonyl)-1H-[1,2,3]-triazol-1-yl]-β-D-galactopyranosyl}-1-naphthyl-carbaldoxim.

In another embodiment the molecule has the general formula shown below:

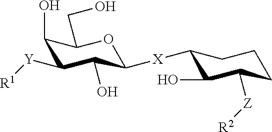

wherein the configuration of the pyranose ring is D-galacto;

X is selected from the group consisting of O, S, and SO;

Y and Z are independently selected from: CONH or a 1H-1,2,3-triazole ring; R¹ and R² are independently selected from the group consisting of: an alkyl group of at least 4 carbons, an alkenyl group of at least 4 carbons, an alkynyl group of at least 4 carbons; a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, and a carbamoyl group substituted with an substituted aryl group; a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group; a phenyl group substituted with at least one trifluoromethoxy group, a phenyl group substituted with at least one sulfo group, a phenyl group substituted with at least one hydroxy group, a phenyl group substituted with at least one carbonyl group, and a phenyl group substituted with at least one substituted carbonyl group; a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, and a heteroaryl group substituted with at least one substituted carbonyl group; and a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group.

In one embodiment Y is CONH.

In another embodiment Y is CONH, wherein the CONH group is linked via the N atom to the pyranose ring.

In a further embodiment Y is a 1H-1,2,3-triazole ring.

In a still further embodiment Y is a 1H-1,2,3-triazole ring, wherein the 1H-1,2,3-triazole ring is linked via the N1 atom to the pyranose ring.

In a further embodiment Z is CONH.

In a still further embodiment Z is CONH, wherein the CONH group is linked via the N atom to the cyclohexane.

In a further embodiment Z is a 1H-1,2,3-triazole ring.

In a still further embodiment Z is a 1H-1,2,3-triazole ring, wherein the 1H-1,2,3-triazole ring is linked via the N1 atom to the cyclohexane.

In a further embodiment R¹ is linked to the C4 atom of the 1H-1,2,3-triazole ring.

In a still further embodiment R¹ is an alkylated carbamoyl group, a fluorinated phenyl group, or a thienyl group.

In a further embodiment R² is linked to the C4 atom of the 1H-1,2,3-triazole ring.

In a still further embodiment R² is an alkylated carbamoyl group, a fluorinated phenyl group, or a thienyl group.

In another embodiment R¹ and R² are independently selected from the group consisting of a carbamoyl group, an alkylated carbamoyl group, an alkenylated carbamoyl group, an arylated carbamoyl group, a phenyl group, a substituted phenyl group, a halogenated phenyl group, a fluorinated phenyl group, a chlorinated phenyl group, a brominated phenyl group, an alkylated phenyl group, an alkenylated phenyl group, a trifluoromethylated phenyl group, a methoxylated phenyl group, a trifluoromethoxylated phenyl group, a naphthyl group, a substituted naphthyl group, a heteroaryl group, a substituted heteroaryl group, a thienyl group, and a substituted thienyl group.

In a further embodiment X is O or S.

In a still further embodiment the molecule is selected from: ((1R,2R,3S)-2-hydroxy-3-(4-(N-(1-propyl)-carbamoyl)-1H-1,2,3-triazol-1-yl)cyclohexyl) 3-deoxy-(3-(4-(N-(1-propyl)-carbamoyl)-1H-1,2,3-triazol-1-yl))-β-D-galactopyranoside; ((1R,2R,3S)-2-hydroxy-3-(4-(2-fluorophenyl)-1H-1,2,3-triazol-1-yl)-cyclohexyl) 3-deoxy-3-(4-(2-fluorophenyl)-1H-1,2,3-triazol-1-yl)-1-thio-β-D-galactopyranoside; ((1R,2R,3S)-2-hydroxy-3-(4-(3-fluorophenyl)-1H-1,2,3-triazol-1-yl)-cyclohexyl) 3-deoxy-3-(4-(3-fluorophenyl)-1H-1,2,3-triazol-1-yl)-1-thio-β-D-galactopyranoside; ((1R,2R,3S)-2-hydroxy-3-(4-(4-fluorophenyl)-1H-1,2,3-triazol-1-yl)-cyclohexyl) 3-deoxy-3-(4-(4-fluorophenyl)-1H-1,2,3-triazol-1-yl)-1-thio-β-D-galactopyranoside; (1R,2R,3S)-2-hydroxy-3-(4-(3-thienyl)-1H-1,2,3-triazol-1-yl)-cyclohexyl) 3-deoxy-3-(4-(3-thienyl)-1H-1,2,3-triazol-1-yl)-1-thio-β-D-galactopyranoside; (1R,2R,3S)-2-hydroxy-3-(4-(N-(1-propyl)-carbamoyl)-1H-1,2,3-triazol-1-yl)-cyclohexyl) 3-deoxy-3-(4-(N-(1-propyl)-carbamoyl)-1H-1,2,3-triazol-1-yl)-1-thio-β-D-galactopyranoside, and (1R,2R,3S)-2-hydroxy-3-(4-chlorobenzamido)-cyclohexyl) 3-deoxy-3-(4-chlorobenzamido)-1-thio-β-D-galactopyranoside.

In a more specific embodiment the molecule has the general formula (13)

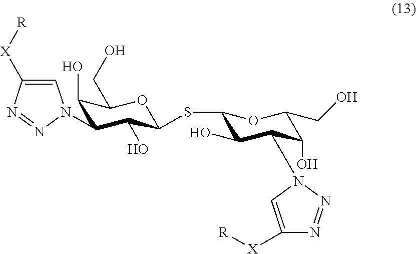

wherein the configuration of at least one of the pyranose rings is D-galacto; X is a bond; R is a phenyl group, which is substituted in any position with one or more substituents selected from the group consisting of methyl, ethyl, isopropyl, tert-butyl, fluoro, chloro, bromo, and trifluoromethyl or R is a thienyl group. Preferably, R is a phenyl group attached to the 1H-1,2,3-triazole ring which phenyl is substituted in any position with one or more substituents selected from the group consisting of fluoro, chloro, and bromo. In a further embodiment R is a phenyl group attached to the 1H-1,2,3-triazole ring which phenyl is substituted in any position with one or more substituents selected from fluoro.

In a further embodiment the configuration of both pyranose rings in formula (13) is D-galacto.

In a still further embodiment the molecule is

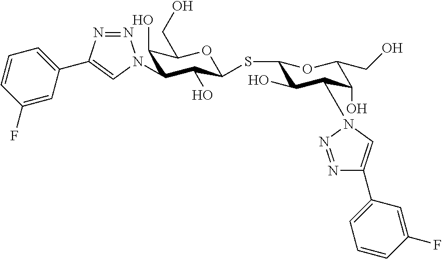

(also referred to herein as TD139), optionally as the free form, such as crystalline form.

In another aspect the present invention relates to a method for treatment or prevention of α-synucleinopathies in a mammalian subject, the method comprising administering a therapeutically effective amount of at least one composition to the subject, wherein the composition comprises a molecule for pharmacological modulation of galectin activity in a mammalian brain.

In one embodiment the galectin activity is galectin-3 activity.

In a further embodiment the pharmacological modulation of galectin activity is inhibition of galectin activity or galectin-3 activity.

In a still further embodiment the method is to treat or prevent a disease or a condition associated with α-synucleinpathies with inflammatory features.

In a further embodiment the disease or condition is selected from a neurodegenerative disease or condition, such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy.

In a still further embodiment the administering comprises contacting the subject or tissue of the subject with a dose of the molecule of at least: about 1 nanograms (ng) to about 100 ng, about 100 ng to about 1000 ng, about 1000 ng to about 2000 ng, about 2000 ng to about 3000 ng, about 3000 ng to about 4000 ng, about 4000 ng to about 5000 ng, about 5000 ng to about 10000 ng, 10000 ng to about 20000 ng, 20000 ng to about 30000 ng, about 30000 ng to about 40000 ng, about 40000 ng to about 60000 ng, about 60000 ng to about 80000 ng, about 100 microgram (μg) to about 500 μg, about 500 μg to about 2000 μg, about 2000 μg to about 4000 μg, about 4000 μg to about 6000 μg, about 6000 μg to about 8000 μg, about 8000 μg to about 10000 μg, about 10000 μg to about 20000 μg, about 20000 μg to about 30000 μg, and about 30000 μg to about 40000 μg.

In a further embodiment the composition is selected from any one of the compositions described in the first aspect and embodiments above.

In a still further embodiment the administering is oral, intravenous, topical, intraperitoneal, nasal, buccal, sublingual, injection into the brain, or subcutaneous administration.

In a further embodiment the composition is in the form of tablets, capsules, powders, nanoparticles, crystals, amorphous substances, solutions, transdermal patches or suppositories.

The publications and other materials, including patents, used herein to illustrate the invention and, in particular, to provide additional details respecting the practice are incorporated by reference in their entirety.

### BRIEF DESCRIPTION OF THE DRAWINGS

FIG. 1. Microglial activation by α-synuclein and inhibition by galectin-3 inhibitor.

We measured iNOS expression by western blot in microglial cells after 12 h incubation with α-synuclein monomers (A) and α-synuclein aggregates (B) using different concentrations, 5 μM, 10 μM and 20 μM. iN-OS was significantly upregulated in with both protein preparations of α-synuclein. α-synuclein aggregates (B) induced a 3-fold higher activation compared to monomers (A). To determine the role of galectin-3 we used a pre-treatment, incubating the galectin-3 inhibitor (TD139) for 30 min and then we incubated for 12 h the cells with α-synuclein, monomers or aggregates, using the highest concentration, 20 μM. The lower iN-OS expression induced by α-synuclein monomers was not significantly inhibited by the galectin-3 inhibitor (TD139) (C). The galectin-3 inhibitor (TD139) significantly inhibited iNOS expression induced by α-synuclein aggregates (D), where 100 μM of (TD139) inhibitor down regulated in iNOS expression, by more than 50%. We use the highest response in each experiment as an internal control to evaluate the response to the other concentrations. (ANOVA) (*P<0.05; * *P<0.005) Error bars, mean±S.E.M (n=3).

FIG. 2. Increased cytokine levels in BV2 microglia culture medium after α-synuclein activation.

Cytokine levels in BV2 microglia culture medium after 12 h incubation with α-synuclein aggregates at concentrations of 5, 10 and 20 μM. α-synuclein aggregates induced a significant increase in cytokine levels of the proinflammatory cytokines TNF-α (A), IL-12 (B) and IL-2 (C). Cytokines levels are represented as pg/μl (ANOVA) (*P<0.05; * *P<0.005; * * * *P<0.0001), Error bars, mean±S.E.M (n=3).

FIG. 3. Galectin-3 siRNA reduce microglial activation induced by α-synuclein aggregates.

BV2 microglia activated by 20 μM of α-synuclein aggregates for 12 h show a robust iNOS downregulation by 80% when galectin-3 is knocked down by siRNA (left). (ANOVA) (*P<0.05) (n=3), Error bars, mean±S.E.M.

FIG. 4. BV2 microglial cells treated with the galectin-3 inhibitor TD139 for 12 h show reduced phagocytic ability.

To test the microglial cell phagocytic ability we studied the ability of microglia to take up fluorescent beads. We used a galectin-3 inhibitor (TD139) (100 μM) as a pre-treatment (30 min) and then incubated the cells with α-synuclein and/or with α-synuclein (20 μM) for 12 h. TD139 robustly inhibited the phagocytosis. By adding galectin-3 we could recover the phagocytic ability even when using the inhibitor at the same time. (n=3) (ANOVA) (*P<0.05; **P<0.005), Error bars, mean±S.E.M.

FIG. 5. Ameliorated proinflammatory response in galectin-3 KO primary microglial cells following α-synuclein activation.

Cytokine levels in culture medium of primary microglial cells was measured after 12 h incubation with α-synuclein aggregates. Treatment of wild type microglia with 5 and 20 μM α-synuclein aggregates for 12 h induced increased levels of IL-1β (A) IL-12 (B), IFN-γ (C) and IL-4 (D). Identical treatment with galectin-3 knock out microglia showed reduced levels of IL-1β (A) IL-12 using 20 μM α-synuclein aggregates (B). Cytokine levels of IFN-γ (C) and IL-4 (D) was not changed in galectin-3 knockout compared to wild type microglia. Electrochemiluminescence ELISA was used to measure the cytokine levels. (ANOVA) (*P<0.05; * *P<0.005) (n=5) Error bars, mean±S.E.M.

FIG. 6. Reduction in IL-8 cytokine release by primary microglial cells from galectin-3 knockout mice.

Cytokine levels in primary microglial cells were analyzed after cells were treated with amyloid-beta fibrils (human recombinant protein, Aβ42). Microglia from galectin-3 knock-out mice demonstrated a significant reduction in cytokine release of IL-8 (65% reduction, P<0.05, n=5) compared to wild-type microglia following challenge by amyloid-beta fibrils at 10 μM Aβ42 or with LPS (1 μg/μl).

FIG. 7. Reduction of IFN-gamma levels in the blood of Alzheimer mice lacking galectin-3.

A remarkable 70% down-regulation of IFN-gamma were found in the blood of Alzheimer mice (5xFAD) lacking galectin-3 (Gal3KO), i.e. Gal3KO/5XFAD (n=6) compared to the normal Alzheimer mice that had galectin-3 present (5xFAD, n=5). In naive wild-type mice (WT), IFN-gamma was barely detectable.

### DETAILED DESCRIPTION

Galectins contain a carbohydrate recognition domain, CRD (Nilsson et al., U.S. patent publication number 2011/0130553 published Jun. 2, 2011, which is incorporated by reference herein in its entirety). The CRD is a tightly folded β-sandwich of about 130 amino acids (about 15 kDa) with the two characteristic features of a β-galactose binding site and sufficient similarity in a sequence motif of about seven amino acids, most of which (about six residues) make up the β-galactose binding site. Further, adjacent sites are required for tight binding of natural saccharides and different preferences of these confer on galectins different fine specificity for natural saccharides.

Completion of human, mouse and rat genome sequences reveal about 15 galectins and galectin-like proteins in one mammalian genome with slight variation between species (Leffler et al. 2004 Glycoconj. J. 19: 433-440; Houzelstein et al. 2004 Mol Biol Evol. 21(7): 1177-1187).

Galectin subunits contain one or two CRDs within a single peptide chain. The first category, mono-CRDs galectins, occurs as monomers or dimers (two types) in vertebrates. Galectin-1 is dimeric and galectin-3 is a monomer in solution and aggregates and becomes multimeric upon encounter with ligands (Leffler et al. 2004 Glycoconj. J. 19: 433-440; Ahmad et al. 2004 J. Biol. Chem. 279: 10841-10847).

Galectins are synthesized as cytosolic proteins on free ribosomes, without a signal peptide. An N-terminus of galectin protein is acetylated, a typical modification of cytosolic proteins, and galectins reside in the cytosol for a long time (not typical of secreted proteins). From cytosol galectins are targeted to the nucleus, specific cytososlic sites, or are secreted (induced or constitutively) by a non-classical (non-ER-Golgi) pathway, as yet unknown, but possibly similar to the export of interleukin-1, IL-1 (Leffler et al. 2004 Glycoconj. J. 19: 433-440).

Amino acid sequences and homology data for galectin proteins are shown in Panjwani, U.S. patent application number 2010/0004163 A1 published Jan. 7, 2010, which is incorporated by reference herein in its entirety.

### Galectin Inhibitor

Herein are provided compositions, methods and kits for treating or preventing a neurodegenerative disease or condition, such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy by administering a galactoside inhibitor of the expression and/or activity of a galectin protein. In certain embodiments a galactoside inhibitor TD139 is used to modulate activity of galectin-3 protein and to treating or preventing a neurodegenerative disease or condition, such as selected from Parkinson's disease. Galectin inhibitors used in various compositions, methods and kits herein for treating or preventing a neurodegenerative disease or condition are found for example in Nilsson et al. U. S. publication number 2004/0147730 A1 (Ser. No. 10/466,933) published Jul. 29, 2004; Nilsson et al. U.S. publication number 2007/0185039 (Ser. No. 11/561,124) published Aug. 7, 2007; Leffler et al. U.S. publication number 2007/0185041 (Ser. No. 11/561,465) published Aug. 9, 2007; Nilsson et al. U.S. publication number 2011/0130553 (Ser. No. 12/992,328) published Jul. 29, 2004; and Leffler et al. U.S. patent publication number 2012/0165277 (Ser. No. 13/266,960) published Jun. 28, 2012, each of which is incorporated by reference herein in its entirety.

Compositions, methods and kits described herein use inhibitors of a galectin protein to selectively treat or prevent a neurodegenerative disease or condition, such as selected from Parkinson's disease without negatively affecting other desired processes in the body.

Galectin-3 has been shown to prolong cell surface residence and thus enhance responsiveness of the TGF-β receptor (Partridge et al. 2004 Science 306: 120-124), which regulates alternative macrophage differentiation into M2 macrophages (MacKinnon et al. 2008. J. Immun. 180: 2650-2658). Galectin-3 has also been shown to play a central role in the recruitment and activation of fibroblasts and thus to formation of scar tissue in various organs, including the kidney, liver and lungs (MacKinnon et al Am J Respir Crit Care Med 2012; 185(5):537-46, Henderson et al Am J Pathol 2008:172; 288, Henderson et al Proc Natl Acad Sci 2006: 103(13); 5060).

Solid phase binding assays and inhibition assays have identified saccharides and glycoconjugates with the ability to bind galectins (reviewed by Leffer et al. 2001 Galectins structure and function—a synopsis (In Mammalian Carbohydrate Recognition Systems Crocker, P., ed., pages 57-83; and Leffler et al. 2004 Glycoconj. J. 19: 433-440). Galectins bind lactose with a dissociation constant (K_{D}) of 0.5-1 mM. K_{D} is the inverse of the association constant, and a lower K_{D} indicates increased binding affinity between molecules. The binding affinity of D-galactose to galectins is generally about 50-fold to 100-fold lower that the binding affinity of lactose to galectins. The binding affinities of N-acetyllactosamine and related disaccharides are variable as these molecules bind a subset of galectins as well as lactose (K_{D} of about 0.5-1 mM), and other galectins about ten-fold less or more than lactose. Small saccharide ligands are effective in binding galectin-3 proteins carrying blood group A-determinants attached to lactose or lacNAc-residues, and were observed to bind about 50-fold greater than the binding affinity for lactose. Galectin-1 shows no preference for these saccharides.

Larger saccharides of the polylactosamine type have been proposed as preferred ligands for galectins such as galectin-3 protein, but not for galectin-1 (Leffler et al. 1986 J. Biol. Chem. 261: 10119-10126). A modified plant pectin polysaccharide has been determined to bind galectin-3 (Pienta et al. 1995 J Natl Cancer Inst. 1995 Mar. 1; 87(5):348-53).

The above-described natural saccharides that have been identified as galectin-3 ligands are not suitable for use as active components in pharmaceutical compositions, because they are susceptible to acidic hydrolysis in the stomach and to enzymatic degradation. In addition, natural saccharides are hydrophilic in nature, and are not readily absorbed from the gastrointestinal tract following oral administration.

### Synthesis of Inhibitors

Saccharides coupled to amino acids with anti-cancer activity are natural compounds in serum, and synthetic analogues have been made (Glinsky et al. 1996 Cancer Res 56: 5319-5324). Saccharides with lactose or galatose coupled to an amino acid inhibit galectins, with about the same potency as the corresponding underivatized sugar. A chemically modified form of citrus pectin (Platt et al. 1992 J. Natl. Cancer. Inst. 84: 438-442) was described as an inhibitor of galectin-3 and as an anti-tumor agent in vivo (Pienta et al., 1995 J. Natl. Cancer Inst. 94:1854-1862).

Natural oligosaccharides, glycoclusters, glycodendrimers, and glycopolymers are too polar and large to be effectively absorbed by the body and in some cases produce immune responses in patients. Furthermore, they are susceptible to acidic hydrolysis in the stomach and to enzymatic hydrolysis.

A thiodigalactoside molecule is synthetic and hydrolytically stable, and is approximately as efficient as N-acetyllactosamine (Leffler et al. 1986 J. Biol. Chem. 261: 10119-10126). A library of pentapeptides was used to obtain low affinity inhibitors of galectin-1 and -3 proteins having similar K_{D} values to that of galactose (Arnusch et al. 2004 Bioorg. Med. Chem. Lett. 14: 1437-1440). Furthermore, peptides are less ideal agents for targeting galectins in vivo, as peptides are susceptible to hydrolysis and are typically polar. N-Acetyllactosamine derivatives carrying aromatic amides or substituted benzyl ethers at C-3′ are highly efficient inhibitors of galectin-3, with IC₅₀ values as low as 4.8 μM, which is a 20-fold improvement in inhibition compared to the natural N-acetyllactosamine disaccharide (Sörme P et al. 2002 Chembiochem. 3(2-3):183-189; and Sörme Pet al. 2003 Methods Enzymol. 363: 157-169). N-Acetyllactosamine derivatives are less polar overall, due to the presence of the aromatic amido moieties and are thus more suitable as agents for the inhibition of galectins in vivo. Furthermore, C3-triazolyl galactosides have been demonstrated to be as potent inhibitors as the corresponding C3-amides of some galectins. Hence, any properly structured galactose C3-substituent may confer enhanced galectin affinity.

C3-amido- and C3-triazolyl-derivatised compounds are susceptible to hydrolytic degradation in vivo, due to the presence of a glycosidic bond in the galactose and N-acetyllactosamine saccharide moiety and, although they are potent small molecule inhibitors of galectin-3, even further improved affinity and stability is desirable. Accordingly, inhibitors based on 3,3′-diamido- or 3,3′-ditriazolyl-derivatization of thiodigalactoside have been developed, (Cumpstey et al. 2005 Angew. Chem. Int. Ed. 44: 5110-5112; Cumpstey et al. 2008 Chem. Eur. J. 14: 4233-4245; and Dam et al. 2008 Biochemistry 47: 8470-8476; International application numbers WO2005113569 and WO2005113568, U.S. patent publication number 2007/185041, and U.S. Pat. No. 7,638,623 B2, each of which is incorporated by reference herein in its entirety) which lack O-glycosidic hydrolytically and enzymatically labile linkages.

However, 3,3′-derivatized thiodigalactosides have disadvantages including a multistep synthesis involving a double inversion reaction to obtain 3-N-derivatized galactose building blocks. Furthermore, cyclohexane replacement of one galactose ring in thiodigalactoside molecules mimics the galactose ring and provides these galectin-1 and -3 inhibitors with efficiency approaching those of the diamido- and ditriazolyl-thiodigalactoside derivatives (International publication number WO 2010/126435 which is incorporated by reference in its entirety). Replacement of a D-galactopyranose unit with a substituted cyclohexane decreases polarity as well as metabolic susceptibility, thus improving drug-like properties.

Known compounds have the general formulas shown below:

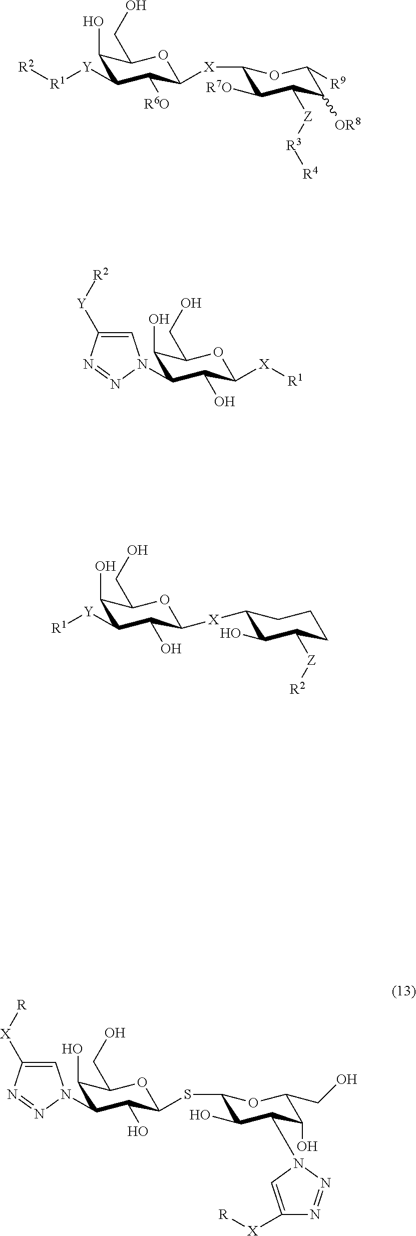

in which in the second structure R¹ can be a D-galactose.

Methods for synthetically preparing galectin protein inhibitors for example for galactosides and intermediates are shown in Nilsson et al. U.S. publication number 2004/0147730 A1 (Ser. No. 10/466,933) published Jul. 29, 2004; Nilsson et al. U.S. publication number 2007/0185039 (Ser. No. 11/561,124) published Aug. 7, 2007; Leffler et al. U.S. publication number 2007/0185041 (Ser. No. 11/561,465) published Aug. 9, 2007; Nilsson et al. U.S. publication number 2011/0130553 (Ser. No. 12/992,328) published Jul. 29, 2004; and Leffler et al. U.S. patent publication number 2012/0165277 (Ser. No. 13/266,960) published Jun. 28, 2012, each of which is incorporated by reference herein in its entirety. Methods for synthesizing the galactosides include reacting a 3-azido-galactosyl thiouronium salt derivative, which is activated to the corresponding thiol in situ, with a 3-azido-galactosyl bromide resulting in the 3,3′-di-azido-thio-di-galactoside.

In certain embodiments, a pharmaceutical composition for use in a method for treatment of Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy includes bis-{3-deoxy-3-[4-(3-fluorophenyl)-1H-1,2,3-triazol-1-yl]-β-D-galactopyranosyl} sulfane, which has a structure shown below:

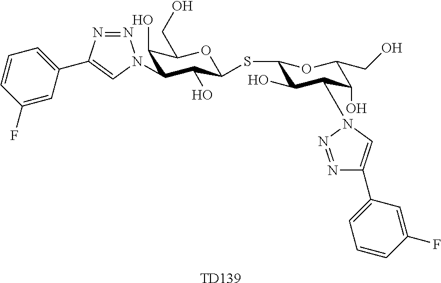

See Mackinnon et al. Am. J. Respir. Crit. Care Med. Mar. 1, 2012 vol. 185 no. 5 537-546, which is incorporated by reference herein in its entirety.

In various embodiments, the molecule is a beta-galactoside, which is derivatized or functionalized, for example, the molecule has the following general formula:

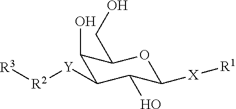

and the configuration of the pyranose ring is D-galacto; X is selected from the group consisting of O, S, NH, CH₂, and NR⁴, or is a bond; Y is selected from the group consisting of NH, CH₂, and NR⁴, or is a bond; R¹ is selected from the group consisting of: a saccharide; hydrogen, an alkyl group, an alkenyl group, an aryl group, a heteroaryl group, and a heterocycle; R² is selected from the group consisting of CO, SO₂, SO, PO, and PO₂; R³ is selected from the group consisting of: an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, and an alkyl group of at least 4 carbon atoms substituted with a halogen; a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxy group, a phenyl group substituted with a carbonyl group and a phenyl group substituted with a substituted carbonyl group; or a phenyl amino group; and R⁴ is selected from the group consisting of hydrogen, an alkyl group, an alkenyl group, an aryl group, a heteroaryl group, and a heterocycle. In various embodiments the composition is non-metabolizable, alternatively the composition in various embodiments is metabolizable.

In certain embodiments, the saccharide (R¹) is selected from the group consisting of glucose, mannose, galactose, N-acetylglucosamine, N-acetylgalactosamine, fucose, fructose, xylose, sialic acid, glucuronic acid, iduronic acid, a disaccharide or, an oligosaccharide comprising at least two of the above saccharides, and derivatives thereof. In certain embodiments, Y is NH, X is O, and the halogen is selected from the group consisting of F, Cl, Br and I.

The molecule in certain embodiments is selected from the group of: methyl 2-acetamido-2-deoxy-4-O-(3-[3-carboxypropanamido]-3-deoxy-β-D-galactopyranosyl)-β-D-glucopyranoside; methyl 2-acetamido-2-deoxy-4-O-(3-[{Z}-3-carboxypropenamido]-3-deoxy-β-D-galactopyranosyl)-β-D-glucopyranoside; methyl 2-acetamido-2-deoxy-4-O-(3-benzamido-3-deoxy-β-D-galactopyranosyl)-β-D-glucopyranoside; methyl 2-acetamido-2-deoxy-4-O-(3-[2-carboxy-benzamido]-3-deoxy-β-D-galactopyranosyl)-β-D-glucopyranoside; methyl 2-acetamido-2-deoxy-4-O-(3-[4-methoxy-2,3,5,6-tetrafluorbenz-amido]-3-deoxy-β-D-galactopyranosyl)-β-D-glucopyranoside; methyl 2-acetamido-2-deoxy-4-O-(3-[2-carboxy-3,4,5,6-tetrafluorbenzamido]-3-deoxy-β-D-galactopyranosyl)-β-D-glucopyranoside; methyl 2-acetamido-2-deoxy-4-O-(3-methanesulfonamido-3-deoxy-β-D-galactopyranosyl)-β-D-glucopyranoside; methyl 2-acetamido-2-deoxy-4-O-(3-[-4-nitrobenzenesulfonamido]-3-deoxy-β-D-galactopyranosyl)-β-D-glucopyranoside, methyl 2-acetamido-2-deoxy-4-O-(3-phenylaminocarbonylam-ino-3-deoxy-β-D-galactopyranosyl)-β-D-glucopyranoside; methyl 2-acetamido-2-deoxy-4-O-(3-aminoacetamido-3-deoxy-β-D-galactopyranosyl)-β-D-glucopyranoside; and methyl 2-acetamido-2-deoxy-4-O-(-3-[{2S}-2-amino-3-carboxy-propanamido]-3-deoxy-β-D-galactopyranosyl)-β-D-glucopyranoside.

The molecule in various embodiments has the general formula:

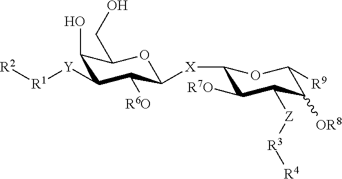

such that the configuration of one of the pyranose rings is β-D-galacto; X is selected from the group consisting of O, S, SO, SO₂, NH, CH₂, and NR⁵, Y is selected from the group consisting of O, S, NH, CH₂, and NR⁵, or is a bond; Z is selected from the group consisting of O, S, NH, CH₂, and NR⁵, or is a bond; R¹ and R³ are independently selected from the group consisting of CO, SO₂, SO, PO₂, PO, and CH₂ or is a bond; and R² and R⁴ are independently selected from the group consisting of: an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; or, a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamnino group, a phenyl group substituted with at least one hydroxy group, a phenyl group substituted with at least one carbonyl group and a phenyl group substituted with at least one substituted carbonyl group; or, a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamnino group, a naphthyl group substituted with at least one hydroxy group, a naphthyl group substituted with at least one carbonyl group and a naphthyl group substituted with at least one substituted carbonyl group; or, a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group. R⁶ and R⁸ are independently selected from the group consisting of a hydrogen, an acyl group, an alkyl group, a benzyl group, and a saccharide. R⁷ is selected from the group consisting of a hydrogen, an acyl group, an alkyl group, and a benzyl group. R⁹ is selected from the group consisting of a hydrogen, a methyl group, hydroxymethyl group, an acyloxymethyl group, an alkoxymethyl group, and a benzyloxymethyl group.

In certain embodiments, Y is NH, Z is NH, X is S, R¹ is CO, R³ is CO, R² or R⁴ is an aromatic for example an aromatic ring; of R⁶, R⁷, and R⁸ is hydrogen; or R⁹ is a hydroxymethyl group. In certain embodiments, the composition is bis-{3-deoxy-3-[4-(3-fluorophenyl)-1H-1,2,3-triazol-1-yl]-β-D-galactopyranosyl} sulfane, bis-(3-deoxy-3-(3-methoxybenzamido)-β-D-galactopyranosyl)sulfane; bis-(3-deoxy-(3,5-dimethoxybenzamido)-β-D-galactopyranosyl)sulfane; bis-(3-deoxy-3-(4-nitrobenzamido)-β-D-galactopyranosyl)sulfane; bis(3-deoxy-3-(2-naphthamido)-β-D-galactopyranosyl)sulfane; bis-(3-deoxy-3-(4-methoxybenzamido)-β-D-galactopyranosyl)sulfane; bis-(3-deoxy-3-(3-nitrobenzamido)-β-D-galactopyranosyl)sulfane; bis-(3-deoxy-3-[4-(dimethylamino)-benzamido]-β-D-galactopyranosyl)sulfane; bis-(3-deoxy-3-(4-methylbenzamido)-β-D-galactopyranosyl)sulfane; bis-(3-deoxy-3-(4-chlorobenzamido)-β-D-galactopyranosyl)sulfane; bis-(3-deoxy-3-(4-tert-butylbenzamido)-β-D-galactopyranosyl)sulfane; bis-(3-deoxy-3-(4-acetylbenzamido)-β-D-galactopyranosyl)sulfane; bis-(3-deoxy-3-[2-(3-carboxy)-naphthamido]-β-D-galactopyranosyl)sulfane; bis-[3-deoxy-3-(3,4-methylenedioxy)benzamido]-β-D-galactopyranosyl)sulfane, bis-(3-deoxy-3-(4-methoxycarbonylbenzamido)-β-D-galactopyranosyl)sulfane; bis-(3-deoxy-3-(3-carboxybenzamido)-β-D-galactopyranosyl)sulfane; bis-(3-deoxy-3-(3-benzyloxy-5-hydroxy-benzamido)-β-D-galactopyranosyl)sulfane; bis-(3-deoxy-3-(3,5-dibenzyloxybenzamido)-β-D-galactopyranosyl)sulfane; bis-(3-deoxy-3-(3-benzyloxy-5-methoxy-benzamido)-β-D-galactopyranosyl)sulfane; bis-(3-deoxy-3-(3-benzyloxy-5-nonyloxy-benzamido)-β-D-galactopyranosyl)sulfane; bis-(3-deoxy-3-(3-hydroxy-5-methoxy-benzamido)-β-D-galactopyranosyl)-sulfane; bis-(3-deoxy-3-(3-hydroxy-5-nonyloxy-benzamido)-β-D-galactopyranosyl)sulfane, bis-(3-deoxy-3-[3-benzyloxy-5-(4-fluoro-benzyloxy)-benzamido]-β-D-galactopyranosyl)sulfane; bis-(3-deoxy-3-[3-methoxy-5-(4-methyl-benzyloxy)-benzamido]-β-D-galactopyranosyl)sulfane; or bis-(3-deoxy-3-(3-allyloxy-5-benzyloxy-benzamido)-β-D-galactopyranosyl)sulfane.

The molecule in various embodiments comprises a 3-triaxolyl-galactoside, for example the composition has a general formula shown below:

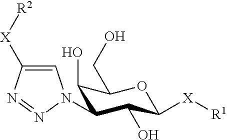

such that the configuration of the pyranose ring is D-galacto; X is selected from the group consisting of O, S, NH, CH₂, and NR⁴, or is a bond; Y is selected from the group consisting of CH₂, CO, SO₂, SO, PO₂ and PO, phenyl, or is a bond; R₁ is selected from the group consisting of: a saccharide; a substituted saccharide; D-galactose; substituted D-galactose; C3-[1,2,3]-triazol-1-yl-substituted D-galactose; a hydrogen, an alkyl group, an alkenyl group, an aryl group, a heteroaryl group, and a heterocycle and derivatives thereof; or an amino group, a substituted amino group, an imino group, or a substituted imino group; and, R² is selected from the group consisting of hydrogen, an amino group, a substituted amino group, an alkyl group, a substituted alkyl group, an alkenyl group, a substituted alkenyl group, an alkynyl group, a substituted alkynyl group, an alkoxy group, a substituted alkoxy group, an alkylamino group, a substituted alkylamino group, an arylamino group, a substituted arylamino group, an aryloxy group, a substituted aryloxy group, an aryl group, a substituted aryl group, a heteroaryl group, a substituted heteroaryl group, and a heterocycle, a substituted heterocycle.

The saccharide in various embodiments is selected from the group consisting of glucose, mannose, galactose, N-acetylglucosamine, N-acetylgalactosamine, fucose, fructose, xylose, sialic acid, glucuronic acid, iduronic acid, galacturonic acid, a disaccharide or an oligosaccharide comprising at least two of the above saccharides, and derivatives thereof.

In various embodiments of the molecule,Y is CO, SO₂, or a bond; R² is an amine or an aryl group; R¹ is galactose, glucose or N-acetylglucosamine; R¹ is substituted galactose, glucose or N-acetylglucosamine; R¹ is a C3-[1,2,3]-triazol-1-yl-substituted galactose; or X is O or S.

In various embodiments of the molecule, R2 is a substituted phenyl group wherein said substituent is one or more selected from the group consisting of halogen, alkoxy, alkyl, nitro, sulfo, amino, hydroxy or carbonyl group.

The molecule in various embodiments is methyl 3-deoxy-3-(1H-[1,2,3]-triazol-1-yl)-1-thio-β-D-galactopyranoside; methyl 3-deoxy-3-(4-propyl-1H-[1,2,3]-triazol-1-yl)-1-thio-β-D-galactopyranoside; methyl 3-(4-methoxycarbonyl-1H-[1,2,3]-triazol-1-yl)-3-deoxy-1-thio-β-D-galactopyranoside; methyl 3-deoxy-3-(4-(1-hydroxy-1-cyclohexyl)-1H-[1,2,3]-triazol-1-yl)-1-thio-β-D-galactopyranoside; methyl 3-deoxy-3-(4-phenyl-1H-[1,2,3]-triazol-1-yl)-1-thio-β-D-galactopyranoside; methyl 3-deoxy-3-(4-p-tolylsulfonyl-1H-[1,2,3]-triazol-1-yl)-1-thio-β-D-gal-actopyranoside; methyl 3-(4-methylaminocarbonyl-1H-[1,2,3]-triazol-1-yl)-3-deoxy-1-thio-β-D-galactopyranoside; methyl 3-(4-butylaminocarbonyl-1H-[1,2,3]-triazol-1-yl)-3-deoxy-1-thio-β-D-galactopyranoside; methyl 3-(4-benzylaminocarbonyl-1H-[1,2,3]-triazol-1-yl)-3-deoxy-1-thio-β-D-galactopyranoside; methyl 3-{4-(3-hydroxyprop-1-ylaminocarbonyl)-1H-[1,2,3]-triazol-1-yl}-3-deoxy-1-thio-β-D-galactopyranoside; methyl 3-{4-[2-(N-morpholino)-ethylaminocarbonyl]-1H-[1,2,3]-triazol-1-yl}-3-deoxy-1-thio-β-D-galactopyranoside; methyl 3-(4-methylaminocarbonyl-1H-[1,2,3]-triazol-1-yl)-3-deoxy-β-D-galactopyranosyl-(1→4)-2-acetamido-2-deoxy-β-D-glucopyranoside, bis-{3-deoxy-3-[4-(3-fluorophenyl)-1H-1,2,3-triazol-1-yl]-β-D-galactopyranosyl} sulfane, methyl 3-deoxy-3-{4-(2-fluorophenyl)-1H-[1,2,3]-triazol-1-yl}-1-thio-β-D-galactopyranoside; methyl 3-deoxy-3-{4-(2-methoxyphenyl)-1H-[1,2,3]-triazol-1-yl}-1-thio-β-D-galactopyranoside; methyl 3-deoxy-3-{4-(3-methoxyphenyl)-1H-[1,2,3]-triazol-1-yl}-1-thio-β-D-galactopyranoside; methyl 3 deoxy-3-{4-(4-methoxyphenyl)-H-[1,2,3]-triazol-1-yl}-1-thio-β-D-galactopyranoside; methyl 3-deoxy-3-{4-(3,5-dimethoxyphenyl)-1H-[1,2,3]-triazol-1-yl}-1-thio-β-D-galactopyranoside; methyl 3-deoxy-3-{4-(1-naphthyl)-1H-[1,2,3]-triazol-1-yl}-1-thio-β-D-galactopyranoside; methyl 3-deoxy-3-{4-(2-naphthyl)-1H-[1,2,3]-triazol-1-yl}-1-thio-β-D-galactopyranoside; methyl 3-deoxy-3-{4-(2-pyridyl)-1H-[1,2,3]-triazol-1-yl}-1-thio-β-D-galactopyranoside; methyl 3-deoxy-3-{4-(3-pyridyl)-1H-[1,2,3]-triazol-1-yl}-1-thio-β-D-galactopyranoside; methyl 3-deoxy-3-{4-(4-pyridyl)-1H-[1,2,3]-triazol-1-yl}-galactopyranoside; O-{3-deoxy-3-[4-phenyl-[1H-[1,2,3]-triazol-1-yl]-β-D-galactopyranosy-1}-3-indol-carbaldoxim; O-{3-deoxy-3-[4-(methylaminocarbonyl)-1H-[1,2,3]-triazol-1-yl]-β-D-galactopyranosyl}-3-indol-carbaldoxim; O-{3-deoxy-3-[4-phenyl-[1H-[1,2,3]-triazol-1-yl]-β-D-galactopyranosy-1}-(2-hydroxy-5-nitro-phenyl)-carbaldoxim; O-{3-deoxy-3-[4-(methylaminocarbonyl)-1H-[1,2,3]-triazol-1-yl]-β-D-galactopyranosyl}-(2-hydroxy-5-nitro-phenyl)-carbaldoxim; O-{3-deoxy-3-[4-phenyl-[1H-[1,2,3]-triazol-1-yl]-β-D-galactopyranosy-1}-(2,5-dihydroxyphenyl)-carbaldoxim; O-{3-deoxy-3-[4-(methylaminocarbonyl)-1H-[1,2,3]-triazol-1-yl]-β-D-galactopyranosyl}-(2,5-dihydroxyphenyl)-carbaldoxim; O-{3-deoxy-3-[4-phenyl-[1H-[1,2,3]-triazol-1-yl]-β-D-galactopyranosy-1}-1-naphthyl-carbaldoxim; or O-{3-deoxy-3-[4-(methylaminocarbonyl)-1H-[1,2,3]-triazol-1-yl]-β-D-galactopyranosyl}-1-naphthyl-carbaldoxim.

The molecule in various embodiments has a thiodigalactoside, for example the molecule has a general formula shown below:

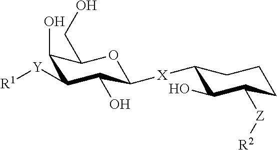

such that the configuration of the pyranose ring is D-galacto; X is selected from the group consisting of O, S, and SO; Y and Z are independently selected from being CONH or a 1H-1,2,3-triazole ring; R¹ and R² are independently selected from the group consisting of: an alkyl group of at least 4 carbons, an alkenyl group of at least 4 carbons, an alkynyl group of at least 4 carbons; a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, and a carbamoyl group substituted with an substituted aryl group; a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group; a phenyl group substituted with at least one trifluoromethoxy group, a phenyl group substituted with at least one sulfo group, a phenyl group substituted with at least one hydroxy group, a phenyl group substituted with at least one carbonyl group, and a phenyl group substituted with at least one substituted carbonyl group; a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, and a heteroaryl group substituted with at least one substituted carbonyl group; and a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group.

The molecule in various embodiment has a formula in which Y is CONH; the CONH group is linked via the N atom to the pyranose ring; the Z is CONH fore example the CONH group is linked via the N atom to the cyclohexane; or Y is a 1H-1,2,3-triazole ring for example the 1H-1,2,3-triazole ring is linked via the N1 atom to the pyranose ring. In various embodiments of the composition, R¹ is linked to the C4 atom of the 1H-1,2,3-triazole ring; Z is a 1H-1,2,3-triazole ring for example the 1H-1,2,3-triazole ring is linked via the N1 atom to the cyclohexane; or R² is linked to the C4 atom of the 1H-1,2,3-triazole ring.

In various embodiments, the molecule includes a digalactoside, for example the molecule includes a general formula (13)

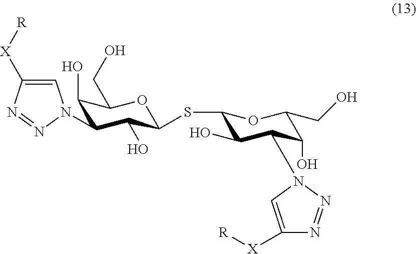

wherein the configuration of at least one of the pyranose rings is D-galacto; X is a bond; R is a phenyl group, which is substituted in any position with one or more substituents selected from the group consisting of methyl, ethyl, isopropyl, tert-butyl, fluoro, chloro, bromo, and trifluoromethyl or R is a thienyl group.

In various embodiments of the molecule R is a phenyl group which is substituted in any position with one or more substituents selected from the group consisting of fluoro, chloro, and bromo. For instance R is a phenyl group which is substituted in any position with one or more substituents selected from fluoro. Typically, the configuration of both pyranose rings is D-galacto.

The term “alkyl group” as used herein includes chemical compounds that consists only of hydrogen and carbon atoms that are bonded by single bonds. For example an alkyl group comprises from about one carbon atom to about seven carbon atoms, and in various embodiments includes about one carbon atom to about four carbon atoms. The alkyl group may be straight- or branched-chain and may also form a cycle comprising from three to seven carbon atoms, such as three, four, five, six, or seven carbon atoms. Thus alkyl refers to any of methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

The term “alkenyl group” as used herein is any functional group or substituent comprising at least one double bond. The alkenyl group includes from about two carbon atoms to about seven carbon atoms. The alkenyl group includes any of vinyl, allyl, but-1-enyl, but-2-enyl, 2,2-dimethylethenyl, 2,2-dimethylprop-1-enyl, pent-1-enyl, pent-2-enyl, 2,3-dimethylbut-1-enyl, hex-1-enyl, hex-2-enyl, hex-3-enyl, prop-1,2-dienyl, 4-methylhex-1-enyl, cycloprop-1-enyl group, and others.

The term “alkylated” as used herein means substituted with an alkyl group. The term “alkenylated” as used herein refers to being substituted with an alkenyl group,

The term “aryl group” as used herein refers to any functional group or substituent derived from an aromatic ring and having about four carbon atoms to about twelve carbon atoms. The aryl group may for example be a phenyl group or a naphthyl group. The above-mentioned groups may be substituted with any other known substituents within the art of organic chemistry. The groups may also be substituted with two or more of the the substituents. Examples of substituents are halogen, alkyl, alkenyl, alkoxy, nitro, sulfo, amino, hydroxy, and carbonyl groups. Halogen substituents are bromo, fluoro, iodo, and chloro. Alkyl groups for example include about one carbon atom to about seven carbon atoms. Alkenyl groups include for example two to seven carbon atoms, such as two carbon atoms or four carbon atoms. Alkoxy groups include one carbon atom to seven carbon atoms, which may contain an unsaturated carbon atom. Combinations of substituents can be present such as trifluoromethyl.

The term “alkoxy group” as used herein is a functional group or substituent including carbon atoms bonded to an oxygen, for example about one carbon atom to about seven carbon atoms. The alkoxy group may be a methoxy group, an ethoxy group, a propoxy group, a isopropoxy group, a n-butoxy group, a sec-butoxy group, tert-butoxy group, pentoxy group, isopentoxy group, 3-methylbutoxy group, 2,2-dimethylpropoxy group, n-hexoxy group, 2-methylpentoxy group, 2,2-dimethylbutoxy group 2,3-dimethylbutoxy group, n-heptoxy group, 2-methylhexoxy group, 2,2-dimethylpentoxy group, 2,3-dimethylpentoxy group, cyclopropoxy group, cyclobutoxy group, cyclopentyloxy group, cyclohexyloxy group, cycloheptyloxy group, and 1-methylcyclopropyl oxy group.

The term “alkylamino group” as used herein is a functional group or substituent including an alkyl group (about one carbon atom to about seven carbon atoms) bond to a nitrogen atom with a lone pair. For example the alkyl group is any of methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

The term “arylamino group” as used herein is an aryl group that is bonded to a derivative of ammonia, such that the derivative of ammonia contains a basic nitrogen atom with a lone pair of electrons. The arylamino group has for example an aryl group having about four carbon atoms to about seven carbon atoms. The arylamino group for example is aniline, carboxylated aniline or halogenated aniline, halogen being as defined above.

The term “aryloxy group” as used herein is an aryl functional group bound to an oxygen. For example the aryloxy group includes about four carbon atoms to about twelve carbon atoms. The aryloxy group may be phenol, carboxylated phenol or halogenated phenol, such that halogen is as defined above. The term “heteroaryl group” as used herein in an aryl group comprising from about four carbon atoms to about 18 carbon atoms, such that at least one atom of the ring is a heteroatom, i.e. not a carbon. In various embodiments, the heteroatom is N, O or S. The heteroaryl group in certain embodiments is a pyridine, or an indole group.

The above-mentioned groups may be substituted with any other known substituents within the art of organic chemistry. The groups may also be substituted with two or more of the substituents. Examples of substituents are halogen, alkoxy, nitro, sulfo, amino, hydroxy, and carbonyl groups. Halogen substituents are bromo, fluoro, iodo, and chloro. In various embodiments, the alkyl groups contain about one carbon atom to about seven carbon atoms. The alkenyl groups in various embodiments include about to two carbon atoms to about seven carbon atoms. The alkoxy in various embodiments includes about one carbon atom to about seven carbon atoms, preferably one to four carbon atoms, and may contain an unsaturated carbon atom.

The term “subject” as used herein refers in various embodiments to mammals and includes humans, primates, livestock animals (e.g., sheep, pigs, cattle, horses, and donkeys), laboratory test animals (e.g., mice, rabbits, rats, and guinea pigs), companion animals (e.g., dogs and cats) and high value zoo and captive wild animals (e.g., foxes, kangaroos, elephants, and deer).

### Polynucleotide Inhibitors

Embodiments of the invention herein, provide a method for treatment or prevention of α-synucleinopathies in a mammalian subject, the method comprising administering a therapeutically effective amount of at least one composition to the subject, wherein the composition comprises a molecule for pharmacological modulation of galectin activity in a mammalian brain. For example, the inhibitor is a recombinantly produced protein administered in situ or ex vivo. The term “recombinant” refers to proteins produced by manipulation of genetically modified organisms, for example micro-organisms or eukaryotic cells in culture.

In an embodiment of the invention, the compositions and methods include a source of the modulator which is an inhibitor such as that a polynucleotide sequences that encode the inhibitory protein, for example the polynucleotide sequence is engineered into recombinant DNA molecules to direct expression of the inhibitory protein or a portion thereof in appropriate host cells. To express a biologically active inhibitor, a nucleotide sequence encoding the inhibitor, or functional equivalent, is inserted into an appropriate expression vector, i.e., a vector that contains the necessary nucleic acid encoding elements that regulate transcription and translation of the inserted coding sequence, operably linked to the nucleotide sequence encoding the amino acid sequence of the inhibitory protein.

Methods that are well known to those skilled in the art are used to construct expression vectors containing a nucleic acid sequence encoding for example a protein or a peptide operably linked to appropriate transcriptional and translational control elements. These methods include in vitro recombinant DNA techniques, synthetic techniques and in vivo recombination or genetic recombination. Techniques are described in Sambrook et al., Molecular Cloning: A Laboratory Manual, Cold Spring Harbor Press, Plainview, N.Y., 1989.

A variety of commercially available expression vector/host systems are useful to contain and express a sequene that encodes a protein or a peptide. These include but are not limited to microorganisms such as bacteria transformed with recombinant bacteriophage, plasmid or cosmid DNA expression vectors; yeast transformed with yeast expression vectors; insect cell systems contacted with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems. See Ausubel et al., Current Protocols in Molecular Biology, John Wiley & Sons, New York, N.Y., 1989.

Virus vectors include, but are not limited to, adenovirus vectors, lentivirus vectors, retrovirus vectors, adeno-associated virus (AAV) vectors, and helper-dependent adenovirus vectors. For example, the vectors deliver a nucleic acid sequence that encodes a transcription factor or agent that binds to a transcription that as shown herein modulates trans-differentation of muscle satellite cells. Adenovirus packaging vectors are commercially available from American Type Tissue Culture Collection (Manassas, Va.). Methods of constructing adenovirus vectors and using adenovirus vectors are shown in Klein et al., Ophthalmology, 114:253-262, 2007 and van Leeuwen et al., Eur. J. Epidemiol., 18:845-854, 2003.

Adenovirus vectors have been used in eukaryotic gene expression (Levrero et al., Gene, 101:195-202, 1991) and vaccine development (Graham et al., Methods in Molecular Biology: Gene Transfer and Expression Protocols 7, (Murray, Ed.), Humana Press, Clifton, N.J., 109-128, 1991). Further, recombinant adenovirus vectors are used for gene therapy (Wu et al., U.S. Pat. No. 7,235,391 issued Jun. 26, 2007).

Recombinant adenovirus vectors are generated, for example, from homologous recombination between a shuttle vector and a provirus vector (Wu et al., U.S. Pat. No. 7,235,391). Helper cell lines for use in these recombinant adenovirus vectors may be derived from human cells such as, 293 human embryonic kidney cells, muscle cells, hematopoietic cells or other human embryonic mesenchymal or epithelial cells. Alternatively, the helper cells may be derived from the cells of other mammalian species that are permissive for human adenovirus, e.g., Vero cells or other monkey embryonic mesenchymal or epithelial cells. Generation and propagation of these replication defective adenovirus vectors using a helper cell line is described in Graham et al, 1997 J. Gen. Virol., 36:59-72, 1977.

Lentiviral vector packaging vectors are commercially available from Invitrogen Corporation (Carlsbad Calif.). An HIV-based packaging system for the production of lentiviral vectors is prepared using constructs in Naldini et al., Science 272: 263-267, 1996; Zufferey et al., Nature Biotechnol., 15: 871-875, 1997; and Dull et al., J. Virol. 72: 8463-8471, 1998.

A number of vector constructs are available to be packaged using a system, based on third-generation lentiviral SIN vector backbone (Dull et al., J. Virol. 72: 8463-8471, 1998). For example the vector construct pRRLsinCMVGFPpre contains a 5′ LTR in which the HIV promoter sequence has been replaced with that of Rous sarcoma virus (RSV), a self-inactivating 3′ LTR containing a deletion in the U3 promoter region, the HIV packaging signal, RRE sequences linked to a marker gene cassette consisting of the Aequora jellyfish green fluorescent protein (GFP) driven by the CMV promoter, and the woodchuck hepatitis virus PRE element, which appears to enhance nuclear export. The GFP marker gene allows quantitation of transfection or transduction efficiency by direct observation of UV fluorescence microscopy or flow cytometry (Kafri et al., Nature Genet., 17: 314-317, 1997 and Sakoda et al., J. Mol. Cell. Cardiol., 31: 2037-2047, 1999).

Manipulation of retroviral nucleic acids to construct a retroviral vector containing a gene that encodes a protein, and methods for packaging in cells are accomplished using techniques known in the art. See Ausubel, et al., 1992, Volume 1, Section III (units 9.10.1-9.14.3); Sambrook, et al., 1989. Molecular Cloning: A Laboratory Manual. Second Edition. Cold Spring Harbor Laboratory Press, Cold Spring Harbor, N.Y.; Miller, et al., Biotechniques. 7:981-990, 1989; Eglitis, et al., Biotechniques. 6:608-614, 1988; U.S. Pat. Nos. 4,650,764, 4,861,719, 4,980,289, 5,122,767, and 5,124,263; and PCT patent publications numbers WO 85/05629, WO 89/07150, WO 90/02797, WO 90/02806, WO 90/13641, WO 92/05266, WO 92/07943, WO 92/14829, and WO 93/14188.

A retroviral vector is constructed and packaged into non-infectious transducing viral particles (virions) using an amphotropic packaging system. Examples of such packaging systems are found in, for example, Miller, et al., Mol. Cell Biol. 6:2895-2902, 1986; Markowitz, et al., J. Virol. 62:1120-1124, 1988; Cosset, et al., J. Virol. 64:1070-1078, 1990; U.S. Pat. Nos. 4,650,764, 4,861,719, 4,980,289, 5,122,767, and 5,124,263, and PCT patent publications numbers WO 85/05629, WO 89/07150, WO 90/02797, WO 90/02806, WO 90/13641, WO 92/05266, WO 92/07943, WO 92/14829, and WO 93/14188.

Generation of “producer cells” is accomplished by introducing retroviral vectors into the packaging cells. Examples of such retroviral vectors are found in, for example, Korman, et al., Proc. Natl. Acad. Sci. USA. 84:2150-2154, 1987; Morgenstern, et al., Nucleic Acids Res. 18:3587-3596, 1990; U.S. Pat. Nos. 4,405,712, 4,980,289, and 5,112,767; and PCT patent publications numbers WO 85/05629, WO 90/02797, and WO 92/07943.

Herpesvirus packaging vectors are commercially available from Invitrogen Corporation, (Carlsbad, Calif.). Exemplary herpesviruses are an α-herpesvirus, such as Varicella-Zoster virus or pseudorabies virus; a herpes simplex virus such as HSV-1 or HSV-2; or a herpesvirus such as Epstein-Barr virus. A method for preparing empty herpesvirus particles that can be packaged with a desired nucleotide segment is shown in Fraefel et al., U.S. Pat. No. 5,998,208, issued Dec. 7, 1999.

The herpesvirus DNA vector can be constructed using techniques familiar to the skilled artisan. For example, DNA segments encoding the entire genome of a herpesvirus is divided among a number of vectors capable of carrying large DNA segments, e.g., cosmids (Evans, et al., Gene 79, 9-20, 1989), yeast artificial chromosomes (YACS) (Sambrook, J. et al., Molecular Cloning: A Laboratory Manual, 2nd Edition, Cold Spring Harbor Press, Cold Spring Harbor, N.Y., 1989) or E. coli F element plasmids (O'Conner, et al., Science 244:1307-1313, 1989).

For example, sets of cosmids have been isolated which contain overlapping clones that represent the entire genomes of a variety of herpesviruses including Epstein-Barr virus, Varicella-Zoster virus, pseudorabies virus and HSV-1. See M. van Zijl et al., J. Virol. 62, 2191, 1988; Cohen, et al., Proc. Nat'l Acad. Sci. U.S.A. 90, 7376, 1993; Tomkinson, et al., J. Virol. 67, 7298, 1993; and Cunningham et al., Virology 197, 116, 1993.

AAV is a dependent parvovirus in that it depends on co-infection with another virus (either adenovirus or a member of the herpes virus family) to undergo a productive infection in cultured cells (Muzyczka, Curr Top Microbiol Immunol, 158:97 129, 1992). For example, recombinant AAV (rAAV) virus is made by co-transfecting a plasmid containing the gene of interest, for example, the Nkx3.2 gene. Cells are also contacted or transfected with adenovirus or plasmids carrying the adenovirus genes required for AAV helper function. Recombinant AAV virus stocks made in such fashion include with adenovirus which must be physically separated from the recombinant AAV particles (for example, by cesium chloride density centrifugation).

Adeno-associated virus (AAV) packaging vectors are commercially available from GeneDetect (Auckland, New Zealand). AAV has been shown to have a high frequency of integration and infects nondividing cells, thus making it useful for delivery of genes into mammalian cells in tissue culture (Muzyczka, Curr Top Microbiol Immunol, 158:97 129, 1992). AAV has a broad host range for infectivity (Tratschin et al., Mol. Cell. Biol., 4:2072 2081, 1984; Laughlin et al., J. Virol., 60(2):515 524, 1986; Lebkowski et al., Mol. Cell. Biol., 8(10):3988 3996, 1988; McLaughlin et al., J. Virol., 62(6):1963 1973, 1988).

Methods of constructing and using AAV vectors are described, for example in U.S. Pat. Nos. 5,139,941 and 4,797,368. Use of AAV in gene delivery is further described in LaFace et al., Virology, 162(2):483 486, 1988; Zhou et al., Exp. Hematol, 21:928 933, 1993; Flotte et al., Am. J. Respir. Cell Mol. Biol., 7(3):349 356, 1992; and Walsh et al., J. Clin. Invest, 94:1440 1448, 1994.

Recombinant AAV vectors have been used for in vitro and in vivo transduction of marker genes (Kaplitt et al., Nat Genet., 8(2):148 54, 1994; Lebkowski et al., Mol. Cell. Biol., 8(10):3988 3996, 1988; Samulski et al., EMBO J., 10:3941 3950, 1991; Shelling and Smith, Gene Therapy, 1: 165 169, 1994; Yoder et al., Blood, 82 (Supp.): 1:347A, 1994; Zhou et al., Exp. Hematol, 21:928 933, 1993; Tratschin et al., Mol. Cell. Biol., 5:3258 3260, 1985; McLaughlin et al., J. Virol., 62(6):1963 1973, 1988) and transduction of genes involved in human diseases (Flotte et al., Am. J. Respir. Cell Mol. Biol., 7(3):349 356, 1992; Ohi et al., Gene, 89(2):279 282, 1990; Walsh et al., J. Clin. Invest, 94:1440 1448, 1994; and Wei et al., Gene Therapy, 1:261 268, 1994).

### Antibody Inhibitors

The present invention in various embodiments includes an inhibitor of a galectin protein for pharmacological modulation of galectin activity. An embodiment of a galectin inhibitor which is a protein includes an antibody that binds to the galectin protein or to a molecule that affects the expression or activity of the galectin protein. The term “antibody” as referred to herein includes whole antibodies and antigen binding fragments (i.e., “antigen-binding portion”) or single chains of these. A naturally occurring “antibody” is a glycoprotein including at least two heavy (H) chains and two light (L) chains interconnected by disulfide bonds.

In various embodiments, an antibody that “specifically binds to a galectin protein” refers to an antibody that binds to a galectin protein with a K_{D} sufficient to inhibit or modulate angiogenesis, for example the K_{D} is 5×10⁻⁹ M or less, 2×10⁻⁹ M or less, or 1×10⁻¹⁰ M or less. For example, the antibody is a monoclonal antibody or a polyclonal antibody. The terms “monoclonal antibody” or “monoclonal antibody composition” as used herein refer to a preparation of antibody molecules of single molecular composition. A monoclonal antibody composition displays a single binding specificity and affinity for a transcription factor or for a particular epitope of a transcription factor. The antibody includes for example an IgM, IgE, IgG such as IgG1 or IgG4.

The terms “polyclonal antibody” or “polyclonal antibody composition” refer to a large set of antibodies each of which is specific for one of the many differing epitopes found in the immunogen, and each of which is characterized by a specific affinity for that epitope. An epitope is the smallest determinant of antigenicity, which for a protein, comprises a peptide of six to eight residues in length (Berzofsky, J. and I. Berkower, (1993) in Paul, W., Ed., Fundamental Immunology, Raven Press, N.Y., p. 246). Affinities range from low, e.g. 10⁻⁶ M to high, e.g., 10⁻¹¹ M. The polyclonal antibody fraction collected from mammalian serum is isolated by well known techniques, e.g. by chromatography with an affinity matrix that selectively binds immunoglobulin molecules such as protein A, to obtain the IgG fraction. To enhance the purity and specificity of the antibody, the specific antibodies may be further purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. Bound antibodies are eluted from the solid phase by standard techniques, such as by use of buffers of decreasing pH or increasing ionic strength, the eluted fractions are assayed, and those containing the specific antibodies are combined.

Also useful for the methods herein is an antibody that is a recombinant antibody. The term “recombinant human antibody”, as used herein, includes antibodies prepared, expressed, created or isolated by recombinant means. Mammalian host cells for expressing the recombinant antibodies used in the methods herein include Chinese Hamster Ovary (CHO cells) including dhfr-CHO cells, described Urlaub and Chasin, Proc. Natl. Acad. Sci. USA 77:4216-4220, 1980 used with a DH FR selectable marker, e.g., as described in R. J. Kaufman and P. A. Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. To produce antibodies, expression vectors encoding antibody genes are introduced into mammalian host cells, and the host cells are cultured for a period of time sufficient to allow for expression of the antibody in the host cells or secretion of the antibody into the culture medium in which the host cells are grown. Antibodies are recovered from the culture medium using standard protein purification methods.

Standard assays to evaluate the binding ability of the antibodies toward the target of various species are known in the art, including for example, an ELISAs, an western blots and an radio immunoassay (RIA). The binding kinetics (e.g., binding affinity) of the antibodies also can be assessed by standard assays known in the art, such as by Biacore analysis.

General methodologies for antibody production, including criteria to be considered when choosing an animal for the production of antisera, are described in Harlow et al. (Antibodies, Cold Spring Harbor Laboratory, pp. 93-117, 1988). For example, an animal of suitable size such as a goat, a dog, a sheep, a mouse, or a camel is immunized by administration of an amount of immunogen, such as the intact protein or a portion thereof containing an epitope from a human transcription factor, effective to produce an immune response. An exemplary protocol involves subcutaneous injection with 100 micrograms to 100 milligrams of antigen, depending on the size of the animal, followed three weeks later with an intraperitoneal injection of 100 micrograms to 100 milligrams of immunogen with adjuvant depending on the size of the animal, for example Freund's complete adjuvant. Additional intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the greatest dilution indicating that having a detectable antibody activity. The antibodies are purified, for example, by affinity purification using binding to columns containing human MAC.

Monoclonal antibodies are generated by in vitro immunization of human lymphocytes. Techniques for in vitro immunization of human lymphocytes are described in Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992. These techniques can be used to produce antigen-reactive monoclonal antibodies, including antigen-specific IgG, and IgM monoclonal antibodies. Any antibody or a fragment thereof having affinity and specific for a transcription factor is within the scope of the modulator molecules provided herein.

The term “antibody” as referred to herein includes whole antibodies and any antigen binding fragment (i.e., “antigen-binding portion”) or single chains thereof, for example, Fv fragments. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to a target (e.g., to a galectin, or a fragment of a galectin, or to a galectin inhibitor, or to a ligand of a galectin in a tissue). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al. 1989 Nature 341:544), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird R. E. et al. 1988 Science 242:423; and Huston, J. S. et al. 1988 Proc Natl Acad Sci USA 85:5879). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities (e.g., an isolated antibody that specifically binds a target such as a galectin, or a fragment of a galectin, or to a galectin inhibitor, or to a ligand of a galectin in a tissue, is substantially free of antibodies that specifically bind antigens other than this target). An isolated antibody that specifically binds DEC205 may, however, have cross-reactivity to other antigens, such as corresponding target molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM, IgE, IgG such as IgG1 or IgG4) that is provided by the heavy chain constant region genes.

The phrases “an antibody recognizing an antigen” and “an antibody specific for an antigen” are used interchangeably herein with the term “an antibody which binds specifically to an antigen.”

As used herein, an antibody or an antibody-fusion protein that specifically binds to a dendritic cell receptor, e.g., to a target which is specifically a human target such as a ligand of a galectin in a human tissue, is intended to refer to an antibody that binds to the human target with a K_{D} of about 5×10⁻⁹ M or less, about 2×10⁻⁹ M or less, or about 1×10⁻¹ M or less. An antibody that “cross-reacts with an antigen other than human target” is intended to refer to an antibody that binds that antigen with a K_{D} of about 0.5×10⁻⁸M or less, about 5×10⁻⁹M or less, or about 2×10⁻⁹ M or less. An antibody that “does not cross-react with a particular antigen” is intended to refer to an antibody that binds to that antigen, with a K_{D} of about 1.5×10⁻⁸M or greater, or a K_{D} of about 5-10×10⁻⁸M or about 1×10⁻⁻⁷M or greater. In certain embodiments, such antibodies that do not cross-react with the antigen exhibit essentially undetectable binding against these proteins in standard binding assays.

As used herein, an antibody that inhibits binding of a target to the galectin refers to an antibody that inhibits a target binding to the receptor with a K of about 1 nM or less, about 0.75 nM or less, about 0.5 nM or less, or about 0.25 nM or less. GL117 is a bacterial anti-β-galactosidase nonspecific isotype-matched rat monoclonal antibody negative control (Hawiger, D. et al. 2001 J Exp Med 194: 769-779).

The term “K_{assoc}” or “Kₐ”, as used herein, is intended to refer to the association rate of a particular antibody-antigen interaction, whereas the term “K_{dis}” or “K_{D},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

As used herein, the term “cross-reactivity” refers to an antibody or population of antibodies binding to epitopes on other antigens. This can be caused either by low avidity or specificity of the antibody or by multiple distinct antigens having identical or very similar epitopes. Cross reactivity is sometimes desirable when one wants general binding to a related group of antigens or when attempting cross-species labeling when the antigen epitope sequence is not highly conserved in evolution.

As used herein, the term “high affinity” for an IgG antibody refers to an antibody having a K_{D} of 10⁻⁸ M or less, 10⁻⁹ M or less, or 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, or 10⁻⁸ M or less.

Standard assays to evaluate the binding ability of the antibodies toward a target of any of various species are known in the art, including for example, ELISAs, western blots and RIAs. Suitable assays are described in detail in the Examples. The binding kinetics (e.g., binding affinity) of the antibodies also can be assessed by standard assays known in the art, such as by Biacore analysis. Assays to evaluate the effects of the antibodies on functional properties of the antibody (e.g., receptor binding, preventing or ameliorating autoimmune disease) are described in further detail in the Examples.

Accordingly, an antibody that “inhibits” one or more of these galectin-related functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (e.g., or when a control antibody of irrelevant specificity is present). An antibody that inhibits a galectin-related activity effects such a statistically significant decrease by at least 10% of the measured parameter, by at least 50%, 80% or 90%, and in certain embodiments an antibody of the invention may inhibit greater than 95%, 98% or 99% of galectin functional activity.

### RNA Interference

Inhibitory agents include RNA interference agents that bind to a nucleic acid that encodes a galectin protein or that encodes a molecule that modulates activity of the galectin protein, such that the nucleic acid modulates angiogenesis. Methods and compositios for binding to the nucleic acid include utilizing RNA interference (RNAi). RNAi is induced by short (e.g., 30 nucleotides) double stranded RNA (dsRNA) molecules which are present in the cell. These short dsRNA molecules, called short interfering RNA (siRNA) cause the destruction of messenger RNAs (mRNAs) which share sequence homology with the siRNA. Beach et al., international publication number WO/2003/062394 published Jul. 31, 2003; McSwiggen et al., U.S. patent publication number 2005/0032733 published Feb. 10, 2005; Cicciarelli et al., U.S. Pat. No. 8,236,771 issued Aug. 7, 2012. In various embodiments, the target nucleic acid sequence encodes a galectin protein or a portion thereof. For example, the RNA interference agent negatively modulates expression of any of galectins 1-11 or a portion thereof (e.g., a carbohydrate binding domain).

Methods for constructing synthetic siRNA or an antisense expression cassette and inserting it into a recombinantly engineered nucleic acid of a vector are well known in the art and are shown for example in Reich et al. U.S. Pat. No. 7,847,090 issued Dec. 7, 2010; Reich et al. U.S. Pat. No. 7,674,895 issued Mar. 9, 2010; Khvorova et al. U.S. Pat. No. 7,642,349 issued Jan. 5, 2010. For example, the invention herein includes synthetic siRNAs that include a sense RNA strand and an antisense RNA strand, such that the sense RNA strand includes a nucleotide sequence substantially identical to a target nucleic acid sequence in cells. Thus, under the circumstances of cells being contacted with viral vectors encoding the siRNAs, the cells express the siRNAs that then negatively modulate expression of the target nucleic acid sequence.

### Galectins

Lectin proteins bind carbohydrates specifically and to agglutinate cells (See international publication number WO/2006/113311 which is incorporated by reference herein in its entirety). Lectins have been shown to be involved in a wide variety of cellular functions including cell-cell and cell-matrix interactions. Lectins are widespread among plants, invertebrates and mammals. Animal lectins have been grouped into families: C-type lectins; P-type lectins; galectins (formerly termed S-type lectins); and pentraxins (see, for example, Barondes et al., J. Biol. Chem. 269:20807, 1994).

Mammalian galectins recognize lactose and related galactosides. While all mammalian galectins share similar affinity for small β-galactosides, they show significant differences in binding specificity for more complex glycoconjugates (Henrick et al., Glycobiology 8:45, 1998; Sato et al., J. Biol. Chem. 267:6983, 1992; and Seetharaman et al., J. Biol. Chem. 273:13047, 1998). In addition to binding β-galactoside sugars, galectins possess hemagglutination activity. Laminin, a naturally occurring glycoprotein containing numerous polylactosamine chains, has been shown to be a natural ligand for certain galectins. Laminin is a component of the basal laminae, the extracellular matrix which underlies all epithelia and surrounds individual muscle, fat and Schwann cells. Interactions between cells and the basal laminae are known to influence the migration and/or differentiation of various cell types during mammalian development. Galectins do not contain traditional sequences that specify membrane translocation, but are both secreted and located intracellularly. In addition to their affinity for β-galactoside sugars, members of the galectin family share significant sequence similarity in the carbohydrate recognition domain (CRD; also referred to as the carbohydrate-binding domain), the relevant amino acid residues of which have been determined by X-ray crystallography (Lobsanov et al., J. Biol. Chem. 267:27034, 1993 and Seetharaman et al., supra). Galectins have been implicated in a wide variety of biological functions including cell adhesion (Cooper et al., J. Cell Biol. 115:1437, 1991), growth regulation (Wells et al., Cell 64:91, 1991), cell migration (Hughes, Curr. Opin. Struct. Biol. 2:687, 1992), neoplastic transformation (Raz et al., Int. J. Cancer 46:871, 1990) and immune responses (Offner et al., J. Neuroimmunol. 28:177, 1990).

### Galectin-1

Galectin-1 forms a homodimer of 14 kilodalton subunits and each subunit has a single binding site. Galectin-1 is synthesized in the cytosol of mammalian cells where the lectin accumulates in a monomeric form (Cummings et al., U.S. Pat. No. 5,948,628 issued Sep. 7, 1999; Cummings et al., U.S. Pat. No. 6,225,071 issued May 1, 2001; Horie et al., U.S. Pat. No. 6,890,531 issued May 10, 2005; and Camby et al, U.S. Pat. No. 7,964,575 issued Jun. 21, 2011).

### Galectin-3

Members of the galectin-3 family of proteins (previously known as CBP-35, Mac-2, L-34, εBP, and RL-29) typically have a sequence of about 240 to 270 amino acids and have molecular weights that from about 25 to about 29 kDa. Galectin-3 proteins are generally composed of a short N-terminal domain, a C-terminal domain which includes a galactoside-binding region, and an intervening proline, glycine, and tyrosine-rich domain which includes repeats of 7-10 conserved amino acids (Liu et al., Biochemistry 35:6073, 1996 and Cherayil et al., Proc. Natl. Acad. Sci. USA, 87:7324, 1990). The tandem repeats are similar to those found in the collagen gene superfamily. The number of repeats varies between galectin-3 proteins and accounts for the differences in size between galectin-3 proteins from different species. The N-terminal domain of galectin-3 permits the protein to undergo multimerization upon binding to surfaces containing glycoconjugate ligands.

Galectin-3 is expressed in various inflammatory cells (e.g., activated macrophages, basophils, and mast cells) and in epithelia and fibroblasts of various tissues (Perillo et al., J. Mol. Med. 76:402, 1998). It is found on the cell surface, within the extracellular matrix (ECM), in the cytoplasm, and in the nucleus of cells. On the cell surface or in the ECM galectin-3 is thought to mediate cell-cell and cell-matrix interactions by binding to complementary glycoconjugates containing polylactosamine chains found in many ECM and cell surface molecules. Galectin-3 is thought to inhibit cell-matrix adhesion by binding to laminin In the nucleus of cells galectin-3 may influence cell-matrix interactions indirectly by influencing the expression of well-known cell adhesion molecules (e.g., α6β1 and α4β7 integrins, Warlfield et al., Invasion Metastasis 17:101, 1997 and Matarrese et al., Int. J. Cancer 85:545, 2000) and cytokines (e.g., IL-1, Jeng et al., Immunol. Lett. 42: 113, 1994). Galectin-3 expression is developmentally regulated in selected organs such as the kidney and its expression level in pulmonary alveolar epithelial cells and hepatocytes is up-regulated following injury. Galectin-3 has been shown to concentrate in the nucleus of certain cell types during proliferation. Expression of galectin-3 is elevated in certain tumors, suggesting galectin-3 plays a role in metastasis. Indeed, overexpression of galectin-3 in a weakly metastatic cell line caused a significant increase in metastatic potential (Raz et al., supra).

Human galectin-3 is 250 amino acids in length and has an approximate molecular weight of 26.1 kDa.

As defined herein, a “galectin-3 protein” includes a galectin-3 “N-terminal domain”, a galectin-3 “proline, glycine, and tyrosine-rich domain”, and/or a galectin-3 “galactoside-binding domain”. These domains are further defined as follows.

As used herein, a galectin-3 “N-terminal domain” includes an amino acid sequence of about 10-20 amino acids, preferably about 14 amino acids that shares at least about 60%, 70%, 80%, 90%, 95%, 99%, or 100% identity with amino acids 1 to 14 of human galectin-3. The N-terminal domain can include an N-glycosylation site (PROSITE No. PS00001) and/or a casein kinase II phosphorylation site (PROSITE No. PS00006). The PROSITE N-glycosylation site has the consensus sequence: N-{P}-[ST]-{P} and the PROSITE casein kinase II phosphorylation site has the consensus sequence: [ST]-X(2)-[DE]. In the above consensus sequences, and other motifs or signature sequences described herein, the standard IUPAC one-letter code for the amino acids is used. Each element in the pattern is separated by a dash (-); square brackets ([ ]) indicate the particular residues that are accepted at that position; X indicates that any residue is accepted at that position; and numbers in parentheses (( )) indicate the number of residues represented by the accompanying amino acid. In certain embodiments, the N-terminal domain includes amino acids L7 and L11 of human galectin-3.

As used herein, a galectin-3 “proline, glycine, and tyrosine-rich domain” includes an amino acid sequence of about 60 to about 140 amino acids, more preferably about 80 to 120 amino acids, or about 90 to 110 amino acids that shares at least about 60%, 70%, 80%, 90%, 95%, 99%, or 100% identity with amino acids 15 to 116 of human galectin-3. The proline, glycine, and tyrosine-rich domain can also include one, two, three, four, five, six, seven, or eight N-myristoylation sites (PROSITE No. PS00008) which have the consensus sequence: G-{EDRKHPFYW}-X(2)-[STAGCN]-{P}. In certain embodiments, the proline, glycine, and tyrosine-rich domain includes the following amino acids and regions of galectin 3: G21, P23, G27, N28, P30, G32, G34, P37, Y41-P46, G53, Y55-G57, P61, G62, G66, P72, G73, G77, Y79-G81, P83, G87, Y89, P90, G99, Y101, P102, P106, Y107, A109, L114, and V116. These amino acids and regions are conserved across several mammalian species of galectin-3 and may play a catalytic and/or structural role.

As used herein, a galectin-3 “galactoside-binding domain” includes an amino acid sequence of about 80 to about 180 amino acids having a bit score for the alignment of the sequence to the consensus sequence PF00337 from PFAM of at least 150. Preferably, a galectin-3 galactoside-binding domain includes at least about 100 to 160 amino acids, more preferably about 110 to 150 amino acids, or about 120 to about 140 amino acids and has a bit score for the alignment of the sequence to the consensus sequence PF00337 from PFAM of at least 150, at least 175, or 200 or greater.

To calculate the bit score for the alignment of a particular sequence to the consensus sequence PF00337 from PFAM, the sequence of interest can be searched against the PFAM database of HMMs (e.g., the PFAM database, release 2.1) using the default parameters available at www.sanger.ac.uk/Software/Pfam. A description of the PFAM database can be found in Sonnhammer et al., supra and a detailed description of HMMs can be found, for example, in Gribskov et al., Meth. Enzymol. 183:146, 1990 and Stultz et al., Protein Sci. 2:305, 1993.

The galectin-3 galactoside-binding domain can further include one, preferably two, protein kinase C phosphorylation sites (PROSITE No. PS00005); a casein kinase II phosphorylation site (PROSITE No. PS00006); and/or a galaptin signature sequence (PROSITE No. PS00309). The protein kinase C phosphorylation site has the following consensus sequence: [ST]-X-[RK]. The galaptin signature sequence has the following consensus sequence: W-[GEK]-X-[EQ]-X-[KRE]-X(3,6)-[PCTF]-[LIVMF]-[NQEGSKV]-X-[GH]-X(3)-[DENKHS]-[LIVMFC]. In certain embodiments, the galectin-3 galactoside-binding domain includes the following amino acids and regions of galectin-3: P117, Y118, L120-L122, G125, P128, R129, L131-1134, G136-V138, N141, N143, R144, L147, F149, R151, G152, D154, A156-F163, E165, R169-N174, N179-G182, E184-R186, F190-E193, G195, P197-K199, Q201-L203, E205, D207-Q220, N222, R224, L228, 1231, 1236, G238-I240, and L242-S244. These amino acids and regions are conserved across several mammalian species of galectin-3 and may play a catalytic and/or structural role.

Certain galectin-3 proteins include the amino acid sequence of human galectin-3. Other galectin-3 proteins include an amino acid sequence that is substantially identical to the amino acid sequence of human galectin-3. The term “substantially identical” is used herein to refer to a first amino acid that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 60%, or 65% identity, preferably at least 75% identity, more preferably at least 85%, 90%, 91%, 92%, 93%, 94%, 95%, 96%, 97%, 98%, or 99% identity to human galectin-3 are termed substantially identical to the amino acid sequence of human galectin-3. In particular, proteins which contain accidentally or deliberately induced alterations, such as deletions, additions, substitutions or modifications of certain amino acid residues of human galectin-3may fall within the definition of galectin-3 proteins provided herein. It will also be appreciated that as defined herein, galectin-3 proteins may include regions represented by the amino acid sequence of galectin-3 taken from other mammalian species including but not limited to bovine, canine, feline, caprine, ovine, porcine, murine, and equine species.

Calculations of sequence identity between sequences are performed as follows. To determine the percent identity of two amino acid sequences, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm In a preferred embodiment, the percent identity between two amino acid sequences is determined using an alignment software program using the default parameters. Suitable programs include, for example, CLUSTAL W by Thompson et al., Nuc. Acids Research 22:4673, 1994 (www.ebi.ac.uk/clustalw), BL2SEQ by Tatusova and Madden, FEMS Microbiol. Lett. 174:247, 1999 (www.ncbi.nlm.nih gov/blast/bl2seq/bl2.html), SAGA by Notredame and Higgins, Nuc. Acids Research 24:1515, 1996 (igs-server.cnrs-mrs.fr/˜cnotred), and DIALIGN by Morgenstern et al., Bioinformatics 14:290, 1998 (bibiserv.techfak.uni-bielefeld.de/dialign).

### Galectin-7

Members of the galectin-7 family of proteins typically exist as monomers that include between about 130 to about140 amino acids and have molecular weights between about 15 and about 16 kDa (see, for example, Magnaldo et al., Develop. Biol. 168:259, 1995 and Madsen et al., J. Biol. Chem. 270:5823, 1995). Expression of galectin-7 has been associated with the onset of epithelial stratification (Timmons et al., Int. J. Dev. Biol. 43:229, 1999). Galectin-7 is thought to play a role in cell-matrix and cell-cell interactions. Galectin-7 is found in areas of cell-cell contact (e.g., in the upper layers of human epidermis); its expression is sharply downregulated in anchorage independent keratinocytes and it is absent in a malignant keratinocyte cell line. Galectin-7 may be required for the maintenance of normal keratinocytes (see, Madsen et al., supra).

Human galectin-7 includes 136 amino acids and has an approximate molecular weight of 15.1 kDa.

As defined herein, a “galectin-7 protein” includes a galectin-7 “galactoside-binding domain”. This domain is further defined as follows.

As used herein, a galectin-7 “galactoside-binding domain” includes an amino acid sequence of about 80 to about 180 amino acids having a bit score for the alignment of the sequence to the consensus sequence PF00337 from PFAM of at least 80. Preferably, a galectin-7 galactoside-binding domain includes at least about 100 to 160 amino acids, or about 110 to 150 amino acids, or about 120 to 140 amino acids and has a bit score for the alignment of the sequence to the consensus sequence PF00337 from PFAM of at least 80, more preferably at least 100, most preferably 120 or greater. The galectin-7 galactoside-binding domain can include one N-glycosylation site (PROSITE No. PS00001); one protein kinase C phosphorylation site (PROSITE No. PS00005); one casein kinase II phosphorylation site (PROSITE No. PS00006); one or two myristoylation sites (PROSITE No. PS00008); and/or a galaptin signature sequence (PROSITE No. PS00309). In certain embodiments, the galectin-7 galactoside-binding domain includes the following amino acids and regions of human galectin-7: M1, S2, H6, K7, L10, P11, G13, R15, G17-V19, R21-G24, V26, P27, A30, R32-Q43, D46-N63, K65, Q67, G68, W70-G76, G78, P80-L90, 192, G97-K99, V101, G103, D104, Y107, H109, F110, H112, R113, P115, V119, R120, V122-L130, S132, I135, and F136. These amino acids and regions are conserved across several mammalian species of galectin-7 and may play a catalytic and/or structural role.

Certain galectin-7 proteins include the amino acid sequence of human galectin-7. Other galectin-7 proteins include an amino acid sequence that is substantially identical to the amino acid sequence of human galectin-7. In particular, proteins which contain accidentally or deliberately induced alterations, such as deletions, additions, substitutions or modifications of certain amino acid residues of human galectin-7 may fall within the definition of galectin-7 herein. It will also be appreciated that as defined herein, galectin-7 proteins may include regions represented by the amino acid sequence of galectin-7 taken from other mammalian species including but not limited to bovine, canine, feline, caprine, ovine, porcine, murine, and equine species.

### Galectin-8

Galectin-8 is a widely expressed protein, present for example, in liver, heart, muscle, kidney, spleen, hind-limb and brain, and the sequence of human and rat galectin-8 genes and proteins are available (see for example Hadari, et al., Trends in Glycosci and Glycotechnol. 9: 103-112, 1997).

Alternative forms of amino acid sequence for human galectin-8 are known for example, a 316 amino acid form (Accession number 000214, created 1 Nov. 1997) and a 359 amino acid form (Accession number Q8TEV1, created 1 Jun. 2002). These sequences, while similar or identical for significant lengths, are not overall mere length variants, having portions of difference.

As defined herein, a “galectin-8 protein” may include a galectin-8 “N-terminal domain”, a galectin-8 “proline, glycine, and tyrosine-rich domain”, and/or a galectin-8 “galactoside-binding domain”. These domains are further defined as follows.

As used herein, a galectin-8 “N-terminal domain” includes an amino acid sequence of about 10-20 amino acids, preferably about 14 amino acids that shares at least about 60%, 70%, 80%, 90%, 95%, 99%, or 100% identity with amino acids 1 to 14 of human galectin-8. The N-terminal domain can include an N-glycosylation site (PROSITE No. PS00001) and/or a casein kinase II phosphorylation site (PROSITE No. PS00006). The PROSITE N-glycosylation site has the consensus sequence: N-{P}-[ST]-{P} and the PROSITE casein kinase II phosphorylation site has the consensus sequence: [ST]-X(2)-[DE]. In the above consensus sequences, and other motifs or signature sequences.

As used herein, a galectin-8 “proline, glycine, and tyrosine-rich domain” includes an amino acid sequence of about 60 to 140 amino acids, more preferably about 80 to 120 amino acids, or about 90 to 110 amino acids that shares at least about 60%, 70%, 80%, 90%, 95%, 99%, or 100% identity with amino acids 15 to 116 of each of human galectin-8. The proline, glycine, and tyrosine-rich domain can also include one, two, three, four, five, six, seven, or eight N-myristoylation sites (PROSITE No. PS00008) which have the consensus sequence: G-{EDRKHPFYW}-X(2)-[STAGCN]-{P}. In certain embodiments, the proline, glycine, and tyrosine-rich domain includes the following amino acids and regions of human galectin-8: G20, P23, P28, G29, G36, P39, and other such residues as are obvious to one of skill in the art. These amino acids and regions are conserved across several mammalian species of galectin-8 and may play a catalytic and/or structural role. In certain embodiments, the proline, glycine, and tyrosine-rich domain includes the following amino acids and regions of galectin-8: G21, P24, P29, G30, G37, P40, and other such residues as are obvious to one of skill in the art.

As used herein, a galectin-4 “galactoside-binding domain” includes an amino acid sequence of about 80 to 180 amino acids having a bit score for the alignment of the sequence to the consensus sequence PF00337 from PFAM of at least 150. Preferably, a galectin-3 galactoside-binding domain includes at least about 100 to 160 amino acids, more preferably about 110 to 150 amino acids, or about 120 to 140 amino acids and has a bit score for the alignment of the sequence to the consensus sequence PF00337 from PFAM of at least 150, more preferably at least 175, most preferably 200 or greater.

To calculate the bit score for the alignment of a particular sequence to the consensus sequence PF00337 from PFAM, the sequence of interest can be searched against the PFAM database of HMMs (e.g., the PFAM database, release 2.1) using the default parameters available at www.sanger.ac.uk/Software/Pfam. A description of the PFAM database can be found in Sonnhammer et al., supra and a detailed description of HMMs can be found, for example, in Gribskov et al., Meth. Enzymol. 183:146, 1990 and Stultz et al., Protein Sci. 2:305, 1993.

A galectin-8 galactoside-binding domain can further include one or two protein kinase C phosphorylation sites (PROSITE No. PS00005); a casein kinase II phosphorylation site (PROSITE No. PS00006); and/or a galaptin signature sequence (PROSITE No. PS00309). The protein kinase C phosphorylation site has the following consensus sequence: [ST]-X-[RK]. The galaptin signature sequence has the following consensus sequence: W-[GEK]-X-[EQ]-X-[KRE]-X(3,6)-[PCTF]-[LIVMF]-[NQEGSKV]-X-[GH]-X(3)-[DENKHS]-[LIVMFC]. In certain embodiments, the galectin-8 galactoside-binding domain includes the following amino acids and regions of human galectin-8: L123-L124, G126, P131, R128, L140-I146, and other sites similar to those as demonstrated above. These amino acids and regions are conserved across several mammalian species of galectin-8 and may play a catalytic and/or structural role.

Certain galectin-8 proteins include the amino acid sequence of human galectin-8. Other galectin-8 proteins include an amino acid sequence that is substantially identical to the amino acid sequence of human galectin-8. The term “substantially identical” is used herein to refer to a first amino acid that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 60%, or 65% identity, preferably at least 75% identity, more preferably at least 85%, 90%, 91%, 92%, 93%, 94%, 95%, 96%, 97%, 98%, or 99% identity to human galectin-8 are termed substantially identical to the amino acid sequence of human galectin-8. In particular, proteins which contain accidentally or deliberately induced alterations, such as deletions, additions, substitutions or modifications of certain amino acid residues of human galectin-8 may fall within the definition of galectin-8 proteins herein. It will also be appreciated that as defined herein, galectin-8 proteins may include regions represented by the amino acid sequence of galectin-8 taken from other mammalian species including but not limited to bovine, canine, feline, caprine, ovine, porcine, murine, and equine species.

### Preparation of Galectin Proteins

It will be appreciated by one of ordinary skill in the art, that the galectins of this invention can be obtained from any available source. These include but are not limited to proteins isolated from natural sources, produced recombinantly or produced synthetically, e.g., by solid phase procedures. In accordance with the present invention, polynucleotide sequences which encode galectin-3, galectin-7 or galectin-8 may be used in recombinant DNA molecules that direct the expression of the galectins of this invention in appropriate host cells. Cherayil et al., supra, Madsen et al., supra, and Hadri et al., supra describe in detail the cloning of human galectin-1, -3, -7 and -8 respectively. In order to express a biologically active galectin-1, galectin-3, galectin-7 or galectin-8, the nucleotide sequence encoding galectin-1, galectin-3, galectin-7, galectin-8 or their functional equivalent, is inserted into an appropriate expression vector, i.e., a vector which contains the necessary elements for the transcription and translation of the inserted coding sequence. Methods which are well known to those skilled in the art can be used to construct expression vectors containing a galectin-1-encoding, galectin-3-encoding, galectin-7-encoding or galectin-8-encoding sequence and appropriate transcriptional or translational controls. These methods include in vitro recombinant DNA techniques, synthetic techniques and in vivo recombination or genetic recombination. The introduction of deletions, additions, or substitutions is achieved using any known technique in the art e.g., using PCR based mutagenesis. Such techniques are described in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. A variety of expression vector/host systems may be utilized to contain and express a galectin-1-encoding, galectin-3-encoding, galectin-7-encoding or galectin-8-encoding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems. Alternatively, the galectins are produced using chemical methods to synthesize a galectin-1, galectin-3, galectin-7 or galectin-8 amino acid sequence, whole or in part. For example, peptide synthesis can be performed using various solid-phase techniques (Roberge et al., Science 269:202, 1995) and automated synthesis may be achieved, for example, using the 431A peptide synthesizer (available from Applied Biosystems of Foster City, Calif.) in accordance with the instructions provided by the manufacturer.

### Pharmaceutical Compositions

In one aspect of the present invention, pharmaceutical compositions are provided, such that these compositions comprise at least one inhibitor of an activity of a galectin protein (e.g., a galectin-1 protein, a galectin-3 protein, a galectin-7 protein, and a galectin-8 protein), and optionally comprise a pharmaceutically acceptable carrier. In certain embodiments, these compositions optionally further comprise one or more additional therapeutic agents.

The phrases “pharmaceutically acceptable carrier” and “pharmaceutically suitable carrier” are used interchangeably herein and include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 discloses various carriers used in formulating pharmaceutical compositions and known techniques for the preparation thereof. Some examples of materials which can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Therapeutically Effective Dose

In yet another aspect, according to the compositions or methods of treatment of the present invention, the method concerns treatment or prevention of α-synucleinopathies in a mammalian subject, the method comprising administering a therapeutically effective amount of at least one composition to the subject, wherein the composition comprises a molecule for pharmacological modulation of galectin activity in a mammalian brain. Thus, the invention provides methods for the treatment of α-synucleinopathies comprising administering a therapeutically effective amount of a pharmaceutical composition comprising active agents that inhibit galectin-3, galectin-7 and/or galectin-8 to a subject in need thereof, in such amounts and for such time as is necessary to achieve the desired result. It will be appreciated that this encompasses administering an inventive pharmaceutical as a therapeutic measure to prevention or treatment of α-synucleinopathies, such as a neurodegenerative disease or condition, such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy. In certain embodiments a “therapeutically effective amount” of the pharmaceutical composition is that amount effective for modulating the galectin activity in the mammalian brain. The compositions, according to the method of the present invention, may be administered using any amount and any route of administration effective for treating the neurodegenerative disease or condition. The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., extent of the neurodegenerative disorder, history of the condition; age, weight and gender of the patient; diet, time and frequency of administration; drug combinations; reaction sensitivities; and tolerance/response to therapy. Long acting pharmaceutical compositions might be administered several times a day, every day, 3 to 4 days, every week, or once every two weeks depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for the patient to be treated. It will be understood, however, that the total daily usage of the compositions will be decided by the attending physician within the scope of sound medical judgment. For any active agent, the therapeutically effective dose can be estimated initially either in cell culture assays or in animal models, usually mice, rabbits, dogs, or pigs. The animal model is also used to achieve a desirable concentration range and route of administration. A therapeutically effective dose refers to that amount of active agent that ameliorates the symptoms or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use.

### Administration of Pharmaceutical Compositions

After formulation with an appropriate pharmaceutically acceptable carrier in a desired dosage, the pharmaceutical compositions of this invention can be administered to humans and other mammals topically, orally, rectally, parenterally, intracisternally, intravaginally, intraperitoneally, subcutanously, intramuscularly, bucally, or nasally, depending on the severity of the condition being treated. Oral administration is envisioned as effective for synthetic small molecule inhibitors.

Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. Administration may be therapeutic or it may be prophylactic.

Powders and sprays can contain, in addition to the agents of this invention, excipients such as talc, silicic acid, aluminum hydroxide, calcium silicates, polyamide powder, or mixtures of these substances. Sprays can additionally contain customary propellants such as HFA.

Transdermal patches have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the molecule in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactidepolyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

### Uses of Pharmaceutical Compositions

As discussed above and described in greater detail in the Examples, an inhibition of at least one of galectin-3, galectin-7 and galectin-8 are useful to treat a neurodegenerative disease or condition, such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy, by binding to other receptors or modulatory proteins or by binding to oligosaccharide chains of secretory mucins to the transmembrane muscins (or other glycoproteins) without being limited by any particular theory or mechanism of action. In general, it is believed that these inhibitors of galectins will be clinically useful in suppressing development of a neurodegenerative disease or condition, such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy.

In general, it is shown herein that these inhibitors of galectins are clinically useful in suppressing a neurodegenerative disease or condition, such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy.

Pharmaceutical compositions containing an inhibitor of at least one of any of galectins 1-11 (e.g., a galectin-1, a galectin-3, a galectin-7 and a galectin-8) are, for example herein, useful to promote α-synucleinopathies.

All animal treatments described in these examples conformed to the Association for Research in Vision and Ophthalmology Resolution on the Use of Animals in Vision Research and the recommendations of the NIH Guide for the Care and Use of Laboratory Animals.

The various embodiments of the invention are exemplified by the following claims and examples and figures are exemplary only and are not to be construed as further limiting.

The contents of all references including non-patent literature references, issued patents and published patent applications cited in this application are hereby incorporated by reference in their entireties.

When the compounds and pharmaceutical compositions herein disclosed are used for the above treatment, a therapeutically effective amount of at least one compound is administered to a mammal in need of said treatment.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The treatment may either be performed in an acute or in a chronic way. The patient to be treated is preferably a mammal; in particular a human being, but it may also include animals, such as dogs, cats, cows, sheep and pigs.

The term “a therapeutically effective amount” of a compound of formula (I) of the present invention as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

In a still further aspect the present invention relates to a pharmaceutical composition comprising the compound of formula (I) and optionally a pharmaceutically acceptable additive, such as a carrier or an excipient.

As used herein “pharmaceutically acceptable additive” is intended without limitation to include carriers, excipients, diluents, adjuvant, colorings, aroma, preservatives etc. that the skilled person would consider using when formulating a compound of the present invention in order to make a pharmaceutical composition.

The adjuvants, diluents, excipients and/or carriers that may be used in the composition of the invention must be pharmaceutically acceptable in the sense of being compatible with the compound of formula (I) and the other ingredients of the pharmaceutical composition, and not deleterious to the recipient thereof. It is preferred that the compositions shall not contain any material that may cause an adverse reaction, such as an allergic reaction. The adjuvants, diluents, excipients and carriers that may be used in the pharmaceutical composition of the invention are well known to a person within the art.

As mentioned above, the compositions and particularly pharmaceutical compositions as herein disclosed may, in addition to the compounds herein disclosed, further comprise at least one pharmaceutically acceptable adjuvant, diluent, excipient and/or carrier. In some embodiments, the pharmaceutical compositions comprise from 1 to 99 weight % of said at least one pharmaceutically acceptable adjuvant, diluent, excipient and/or carrier and from 1 to 99 weight % of a compound as herein disclosed. The combined amount of the active ingredient and of the pharmaceutically acceptable adjuvant, diluent, excipient and/or carrier may not constitute more than 100% by weight of the composition, particularly the pharmaceutical composition.

In some embodiments, only one compound as herein disclosed is used for the purposes discussed above.

In some embodiments, two or more of the compound as herein disclosed are used in combination for the purposes discussed above.

The composition, particularly pharmaceutical composition comprising a compound set forth herein may be adapted for oral, intravenous, topical, intraperitoneal, nasal, buccal, sublingual, or subcutaneous administration, or for administration via the respiratory tract in the form of, for example, an aerosol or an air-suspended fine powder. Therefore, the pharmaceutical composition may be in the form of, for example, tablets, capsules, powders, nanoparticles, crystals, amorphous substances, solutions, transdermal patches or suppositories.

The composition and particularly pharmaceutical composition may optionally comprise two or more compounds of the present invention. The composition may also be used together with other medicaments within the art for the treatment of related disorders.

The typical dosages of the compounds set forth herein vary within a wide range and depend on many factors, such as the route of administration, the requirement of the individual in need of treatment, the individual's body weight, age and general condition.

The compound of formula (I) may be prepared as described in the experimental section below.

Further embodiments of the process are described in the experimental section herein, and each individual process as well as each starting material constitutes embodiments that may form part of embodiments.

The above embodiments should be seen as referring to any one of the aspects (such as ‘method for treatment’, ‘pharmaceutical composition’, ‘compound for use as a medicament’, or ‘compound for use in a method’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless other-wise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### REFERENCES

Blandini F (2013) Neural and immune mechanisms in the pathogenesis of Parkinson's disease. Journal of neuroimmune pharmacology: the official journal of the Society on NeuroImmune Pharmacology 8:189-201.
Casey G (2013) Parkinson's disease: a long and difficult journey. Nursing New Zealand 19:20-24.
Codolo G, Plotegher N, Pozzobon T, Brucale M, Tessari I, Bubacco L, de Bernard M (2013) Triggering of inflammasome by aggregated alpha-synuclein, an inflammatory response in synucleinopathies. PloS one 8:e55375.
De Pablos R M, Herrera A J, Villaran R F, Cano J, Machado A (2005) Dopamine-dependent neurotoxicity of lipopolysaccharide in substantia nigra. FASEB journal: official publication of the Federation of American Societies for Experimental Biology 19:407-409.
Deierborg T (2013) Preparation of primary microglia cultures from postnatal mouse and rat brains. Methods in molecular biology 1041:25-31.
Fellner L, Irschick R, Schanda K, Reindl M, Klimaschewski L, Poewe W, Wenning G K, Stefanova N (2013) Toll-like receptor 4 is required for alpha-synuclein dependent activation of microglia and astroglia. Glia 61:349-360.
George J M, Jin H, Woods W S, Clayton D F (1995) Characterization of a novel protein regulated during the critical period for song learning in the zebra finch. Neuron 15:361-372.
Ghee M, Melki R, Michot N, Mallet J (2005) PA700, the regulatory complex of the 26S proteasome, interferes with alpha-synuclein assembly. The FEBS journal 272:4023-4033.
Henn A, Lund S, Hedtjarn M, Schrattenholz A, Porzgen P, Leist M (2009) The suitability of BV2 cells as alternative model system for primary microglia cultures or for animal experiments examining brain inflammation. Altex 26:83-94.
Jiang H R, Al Rasebi Z, Mensah-Brown E, Shahin A, Xu D, Goodyear C S, Fukada S Y, Liu FT, Liew F Y, Lukic M L (2009) Galectin-3 deficiency reduces the severity of experimental autoimmune encephalomyelitis. Journal of immunology 182:1167-1173.
Karlsson A, Christenson K, Matlak M, Bjorstad A, Brown K L, Telemo E, Salomonsson E, Leffler H, Bylund J (2009) Galectin-3 functions as an opsonin and enhances the macrophage clearance of apoptotic neutrophils. Glycobiology 19:16-20.
Kholodilov N G, Neystat M, Oo T F, Lo S E, Larsen K E, Sulzer D, Burke R E (1999) Increased expression of rat synuclein in the substantia nigra pars compacta identified by mRNA differential display in a model of developmental target injury. Journal of neurochemistry 73:2586-2599.
Kim C, Ho D H, Suk J E, You S, Michael S, Kang J, Joong Lee S, Masliah E, Hwang D, Lee H J, Lee S J (2013) Neuron-released oligomeric alpha-synuclein is an endogenous agonist of TLR2 for paracrine activation of microglia. Nature communications 4:1562.
Kruger R, Kuhn W, Muller T, Woitalla D, Graeber M, Kosel S, Przuntek H, Epplen J T, Schols L, Riess O (1998) Ala30Pro mutation in the gene encoding alpha-synuclein in Parkinson's disease. Nat Genet 18:106-108.
Lalancette-Hebert M, Swamp V, Beaulieu J M, Bohacek I, Abdelhamid E, Weng Y C, Sato S, Kriz J (2012) Galectin-3 Is Required for Resident Microglia Activation and Proliferation in Response to Ischemic Injury. Journal of Neuroscience 32:10383-10395.
Larson M E, Sherman M A, Greimel S, Kuskowski M, Schneider J A, Bennett D A, Lesne S E (2012) Soluble alpha-synuclein is a novel modulator of Alzheimer's disease pathophysiology. The Journal of neuroscience: the official journal of the Society for Neuroscience 32:10253-10266.
Lee H J, Bae E J, Lee S J (2014) Extracellular alpha-synuclein-a novel and crucial factor in Lewy body diseases. Nature reviews Neurology.
Lee H J, Suk J E, Bae E J, Lee S J (2008) Clearance and deposition of extracellular alpha-synuclein aggregates in microglia. Biochemical and biophysical research communications 372:423-428.
Li A, Choi Y S, Dziema H, Cao R, Cho H Y, Jung Y J, Obrietan K (2010) Proteomic profiling of the epileptic dentate gyms. Brain pathology 20:1077-1089.
Liu F T, Rabinovich G A (2010) Galectins: regulators of acute and chronic inflammation. Ann N Y Acad Sci 1183:158-182.
Lu J Q, Fan Y, Mitha A P, Bell R, Metz L, Moore G R, Yong V W (2009) Association of alpha-synuclein immunoreactivity with inflammatory activity in multiple sclerosis lesions. Journal of neuropathology and experimental neurology 68:179-189.
Lyman M, Lloyd D G, Ji X, Vizcaychipi M P, Ma D (2014) Neuroinflammation: The role and consequences. Neuroscience research 79C:1-12.
Mackinnon A C, Gibbons M A, Farnworth S L, Leffler H, Nilsson U J, Delaine T, Simpson A J, Forbes S J, Hirani N, Gauldie J, Sethi T (2012) Regulation of transforming growth factor-betal-driven lung fibrosis by galectin-3. American journal of respiratory and critical care medicine 185:537-546.
Mondello S, Buki A, Italiano D, Jeromin A (2013) alpha-Synuclein in CSF of patients with severe traumatic brain injury. Neurology 80:1662-1668.
Pajoohesh-Ganji A, Knoblach S M, Faden A I, Byrnes K R (2012) Characterization of inflammatory gene expression and galectin-3 function after spinal cord injury in mice. Brain research 1475:96-105.
Qiao S, Luo J H, Jin J H (2012) [Role of microglial activation induced by alpha-synuclein in pathogenesis of Parkinson's disease]. Zhejiang da xue xue bao Yi xue ban=Journal of Zhejiang University Medical sciences 41:210-214.
Rey N L, Petit G H, Bousset L, Melki R, Brundin P (2013) Transfer of human alpha-synuclein from the olfactory bulb to interconnected brain regions in mice. Acta Neuropathol 126:555-573.
Riku Y, Ikeuchi T, Yoshino H, Mimuro M, Mano K, Goto Y, Hattori N, Sobue G, Yoshida M (2013) Extensive aggregation of alpha-synuclein and tau in juvenile-onset neuroaxonal dystrophy: an autopsied individual with a novel mutation in the PLA2G6 gene-splicing site. Acta neuropathologica communications 1:12.
Rojanathammanee L, Murphy E J, Combs C K (2011) Expression of mutant alpha-synuclein modulates microglial phenotype in vitro. Journal of neuroinflammation 8:44.
Saksida T, Nikolic I, Vujicic M, Nilsson U J, Leffler H, Lukic M L, Stojanovic I, Stosic-Grujicic S (2013) Galectin-3 deficiency protects pancreatic islet cells from cytokine-triggered apoptosis in vitro. Journal of cellular physiology 228:1568-1576.
Samii A, Nutt J G, Ransom B R (2004) Parkinson's disease. The Lancet 363:1783-1793.
Sano H, Hsu D K, Apgar J R, Yu L, Sharma B B, Kuwabara I, Izui S, Liu F T (2003) Critical role of galectin-3 in phagocytosis by macrophages. The Journal of clinical investigation 112:389-397.
Shin T (2013) The pleiotropic effects of galectin-3 in neuroinflammation: a review. Acta Histochem 115:407-411.
Spillantini M G, Schmidt M L, Lee V M, Trojanowski J Q, Jakes R, Goedert M (1997) Alpha-synuclein in Lewy bodies. Nature 388:839-840.
Stefanis L (2012) alpha-Synuclein in Parkinson's disease. Cold Spring Harbor perspectives in medicine 2:a009399.
Stirling D P, Cummins K, Mishra M, Teo W, Yong V W, Stys P (2013) Toll-like receptor 2-mediated alternative activation of microglia is protective after spinal cord injury. Brain: a journal of neurology.
Tomas-Camardiel M, Rite I, Herrera A J, de Pablos R M, Cano J, Machado A, Venero J L (2004) Minocycline reduces the lipopolysaccharide-induced inflammatory reaction, peroxynitrite-mediated nitration of proteins, disruption of the blood-brain barrier, and damage in the nigral dopaminergic system. Neurobiology of disease 16:190-201.
Unal-Cevik I, Gursoy-Ozdemir Y, Yemisci M, Lule S, Gurer G, Can A, Muller V, Kahle P J, Dalkara T (2011) Alpha-synuclein aggregation induced by brief ischemia negatively impacts neuronal survival in vivo: a study in [A30P]alpha-synuclein transgenic mouse. Journal of cerebral blood flow and metabolism: official journal of the International Society of Cerebral Blood Flow and Metabolism 31:913-923.
Villar-Cheda B, Dominguez-Meijide A, Joglar B, Rodriguez-Perez A I, Guerra M J, Labandeira-Garcia J L (2012) Involvement of microglial RhoA/Rho-kinase pathway activation in the dopaminergic neuron death. Role of angiotensin via angiotensin type 1 receptors. Neurobiology of disease 47:268-279.
Volarevic V, Milovanovic M, Ljujic B, Pejnovic N, Arsenijevic N, Nilsson U, Leffler H, Lukic M L (2012) Galectin-3 deficiency prevents concanavalin A-induced hepatitis in mice. Hepatology 55:1954-1964.
Yang R Y, Hsu D K, Liu F T (1996) Expression of galectin-3 modulates T-cell growth and apoptosis. Proc Natl Acad Sci USA 93:6737-6742.
Zhao Z, Wang J, Zhao C, Bi W, Yue Z, Ma Z A (2011) Genetic ablation of PLA2G6 in mice leads to cerebellar atrophy characterized by Purkinje cell loss and glial cell activation. PloS one 6:e26991.
Zindler E, Zipp F (2010) Neuronal injury in chronic CNS inflammation. Best practice & research Clinical anaesthesiology 24:551-562.

### Experimental

### EXAMPLE 1

### Materials and Instruments for Synthesis of bis-{-deoxy-3-[4-(3-fluorophenyl)-1H-1,2,3-triazol-1-yl]-β-D-galactopyranosyl} sulfane

Bis-{3-deoxy-3-[4-(3-fluorophenyl)-1H-1,2,3-triazo1-1-yl]-β-D-galactopyranosyl} sulfane (TD139) was provided by Profs. Hakon Leffler and Ulf Nilsson (Lund University), and was prepared using the materials and methods described herein.

Melting points were recorded on a Kofler apparatus (Reichert) and are uncorrected. Proton nuclear magnetic resonance (1H) spectra were recorded using a Bruker DRX 400 (400 MHz) or a Bruker ARX 300 (300 MHz) spectrometer; multiplicities are quoted as singlet (s), doublet (d), doublet of doublets (dd), triplet (t), apparent triplet (at) or apparent triplet of doublets (atd). Carbon nuclear magnetic resonance (13C) spectra were recorded using a Bruker DRX 400 (100.6 MHz) spectrometer. Spectra were assigned using COSY, HMQC and DEPT experiments. All chemical shifts are quoted on the d-scale in parts per million (ppm).

Low- and high-resolution (FAB-HRMS) fast atom bombardment mass spectra were recorded using a JEOL SX-120 instrument and low- and high- resolution (ES-HRMS) were recorded with a Micromass Q-TOF instrument. Optical rotations were measured on a Perkin-Elmer 341 polarimeter with a path length of 1 dm; concentrations are given in g per 100 mL. Thin layer chromatography (TLC) was performed using Merck Kieselgel sheets, pre-coated with 60F254 silica. Plates were developed using 10% sulfuric acid. Flash column chromatography was performed with silica (Matrex, 60 Å, 35-70 μm, Grace Amicon). Acetonitrile was distilled from calcium hydride and stored over 4 Å molecular sieves. DMF was distilled from 4 Å molecular sieves and stored over 4 Å molecular sieves.

Bis-{3-deoxy-3-[4-(3-fluorophenyl)-1H-1,2,3-triazol-1-yl]-β-D-galactopyranosyl} sulfane (TD139) was prepared in accordance with the reaction scheme 1 below:

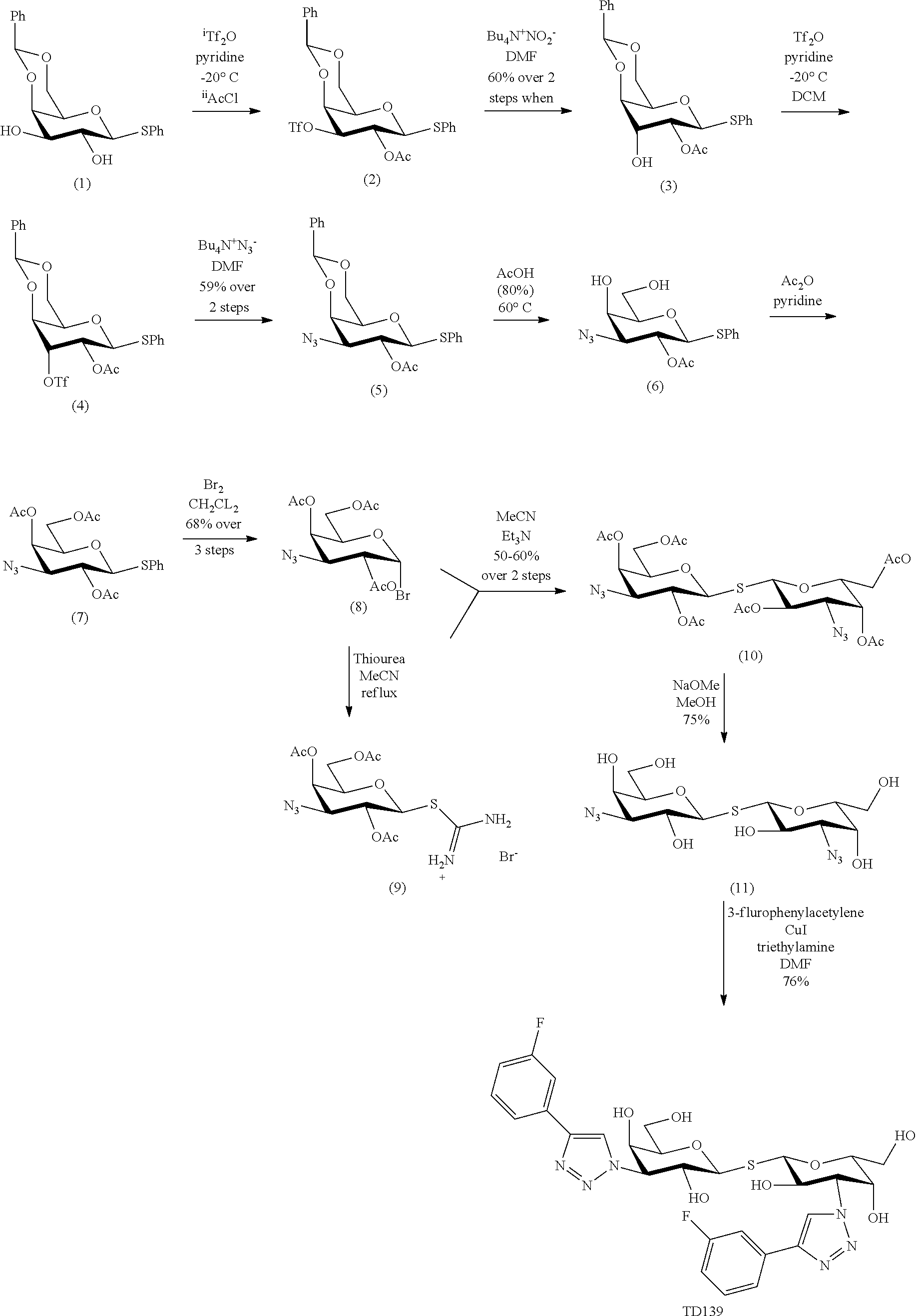

Compound 1 (reaction 1 above) was obtained from Carbosynth Limited 8 & 9 Old Station Business Park—Compton—Berkshire—RG20 6NE—UK or synthesized in three near-quantitative steps from D-galactose, (see Li, Z. and Gildersleeve, J. J. Am. Chem. Soc. 2006, 128, 11612-11619).

### EXAMPLE 2

### Synthesis of Phenyl 2-O-acetyl-4,6-O-benzylidene-1-thio-3-O-trifluoromethanesulfonyl-β-D-galactopyranoside (Structure 2 in Scheme 1)

Compound 1 (10.5 grams, 29.2 mmol) was dissolved in dried pyridine (4.73 mL, 58 4 mmol) and dried CH₂Cl₂ (132 mL). The reaction mixture was cooled, with stirring, to −20° C. (ice and NaCl bath 3:1). Slowly and under N₂ atmosphere, Tf₂O (5.68 mL, 33.6 mmol) was added. The reaction mixture was monitored by TLC (heptane:EtOAc, 1:1 and toluene:acetone, 10:1). When the reaction was complete, AcCl (2.29 mL, 32.1 mmol) was added and stirring was maintained, and the temperature was increased to room temperature. This mixture was monitored by TLC (heptane:EtOAc, 1:1 and toluene:acetone, 10:1). When the reaction was complete, it was quenched with CH₂Cl₂ and washed with 5% HCl, NaHCO₃ (saturated) and NaCl (saturated). The organic layer was dried over MgSO₄, filtered and concentrated under reduced pressure.

### EXAMPLE 3

### Synthesis of phenyl 2-O-acetyl-4,6-O-benzyliden-1-thio-β-D-gulopyranoside (Structure 3 in Scheme 1)

Tetrabutylammonium nitrite (25.3 g, 87.7 mmol) was added to a solution of compound 2 (15.6 g, 29.2 mmol) in DMF (110 mL) and was kept stirring, under N₂ atmosphere, at 50° C. The reaction was observed initially to have a purple color which later was observed to be garnet colored. The reaction was monitored by TLC (heptane:EtOAc, 1:1 and toluene:acetone, 10:1) and quenched with CH₂Cl₂. The mixture was washed with 5% HCl, NaHCO₃ (saturated) and NaCl (saturated). The organic layer was dried with MgSO₄, and was filtered and concentrated under reduced pressure followed by purification by flash chromatography (eluent heptane:EtOAc, 1:1 and heptane:EtOAc, 1:2) and recrystallized from a mixture of EtOAc and heptane (1:3). ¹H NMR in CDCl₃ δ 7.60-7.57 (m, 2H, Ar), 7.43-7.40 (m, 2H, Ar), 7.37-7.34 (m, 3H, Ar), 7.29-7.25 (m, 3H, Ar), 5.50 (s, 1H, PhCH), 5.15 (d, 1H, J=10.29 Hz, H-1), 5.10 (dd, 1H, J=10.27 Hz, 2.85 Hz, H-2), 4.36 (dd, 1H, J=12.49 Hz,1.4 Hz, H-6), 4.18 (br s, 1H, H-3), 4.08 (dd, 1H, J=3.59 Hz, 1.04 Hz, H-6), 4.03 (dd, 1H, J=12.53 Hz, 1.75 Hz, H-4), 3.88 (s, 2H, H-5+OH), 2.12 (s, 3H, OAc).

### EXAMPLE 4

### Synthesis of phenyl 2-O-acetyl-4,6-O-benzylidene-1-thio-3-O-trifluoromethanesulfonyl-β-D-gulopyranoside (Structure 4 in Scheme 1)

Compound 3 (1.00 g, 2.48 mmol) was dissolved in dried CH₂Cl₂ (12.5 mL) and dried pyridine (0.40 mL, 4.96 mmol). The reaction mixture was cooled, with stirring, to −20° C. (ice and NaCl bath 3:1). Slowly and under N₂ atmosphere, Tf₂O (0.48 mL, 2.85 mmol) was added. The reaction mixture was monitored by TLC (heptane:EtOAc, 1:1 and toluene:acetone, 10:1) and when complete, was quenched with CH₂Cl₂ and washed with 5% HCl, NaHCO₃ (saturated) and NaCl (saturated). The organic layer was dried over MgSO₄, and was filtered and concentrated under reduced pressure to dryness.

### EXAMPLE 5

### Synthesis of phenyl 2-O-acetyl-3-azido-4,6-O-benzylidene-3-deoxy-1-thio-β-D-galactopyranoside (Structure 5 in Scheme 1)

Tetrabutylammonium azide (2.12 g, 7.44 mmol) was added carefully to a solution of compound 4 (1.3256 g, 2.48 mmol) in DMF (10 mL) with stirring, under N₂ atmosphere, at 50° C. The reaction was monitored by TLC (E:H, 1:1) and concentrated under reduced pressure followed by purification by flash chromatography (eluent heptane:EtOAc, 2:1 and heptane:EtOAc, 1:1). ¹H NMR in CDCl₃ δ 7.61-7.58 (m, 2H, Ar), 7.44-7.41 (m, 2H, Ar), 7.39-7.36 (m, 3H, Ar), 7.30-7.24 (m, 3H, Ar), 5.59 (s, 1H, PhCH), 5.35 (t, 1H, J=9.95 Hz, H-2), 4.73 (d, 1H, J=9.63 Hz, H-1), 4.44 (dd, 1H, J=6.24 Hz, 1.60 Hz, H-6), 4.35-4.34 (dd, 1H, J=3.33 Hz, 0.88 Hz, H-4), 4.11 (dd, 1H, J=12.48 Hz, 1.67 Hz, H-6), 3.57 (d, 1H, J=1.15 Hz, H-5), 3.44 (dd, 1H, J=10.21 Hz, 3.29 Hz, H-3), 2.17 (s, 3H, OAc).

### EXAMPLE 6

### Synthesis of phenyl 2-O-acetyl-3-azido-3-deoxy-1-thio-β-D-galactopyranoside (Structure 6 in Scheme 1)

Compound 5 (470 mg, 1.1 mmol) was dissolved in 80% acetic acid (75 mL) and the mixture was heated and maintained at 60° C. The reaction was monitored by TLC (heptane:EtOAc, 1:1). When the reaction was complete, the mixture was concentrated under reduced pressure with heat.

### EXAMPLE 7

### Synthesis of phenyl 2,4,6-tri-O-acetyl-3-azido-3-deoxy-1-thio-β-D-galactopyranoside (Structure 7 in Scheme 1)

Acetic anhydride (30 mL) was added to a solution of compound 6 (373 mg, 1.1 mmol) in dry pyridine (30 mL). The reaction was monitored by TLC (heptane:EtOAc, 1:1) and when complete, was concentrated under reduced pressure. ¹H NMR in CDCl₃ δ 7.54-7.51 (m, 2H, Ar), 7.35-7.30 (m, 3H, Ar), 5.46 (dd, 1H, H-4), 5.23 (t, 1H, H-2), 4.73 (d, 1H, H-1), 4.15 (d, 2H, H-6, H-6), 3.94 (dt, 1H, H-5), 3.68 (dd, 1H, H-3), 2.18 (s, 3H, OAc), 2.15 (s, 3H, OAc), 2.06 (s, 3H, OAc).

### EXAMPLE 8

### Synthesis of 2,4,6-tri-O-acetyl-3-azido-3-deoxy-α-D-galactopyranosyl bromide (Structure 8 in Scheme 1)

Compound 7 (237.4 mg, 560 μmol) was dissolved in dry CH₂Cl₂ (2 mL), and bromine (32 μl, 620 μmol) was added. The reaction was monitored by TLC (heptane:EtOAc, 1:1). When complete, a small amount of cyclopentene was added to the reaction mixture to remove remaining untreated Br₂. The mixture was concentrated under reduced pressure and purified by quick Flash chromatography (eluent: 500 mL heptane:EtOAc, 2:1).

### EXAMPLE 9

### Synthesis of 2,4,6-tri-O-acetyl-3-azido-3-deoxy-α-D-galactopyranose-1-isothiouronium bromide (Structure 9 in Scheme 1)

The sensitive bromide compound 8 (70.6 mg, 180 μmol) was immediately dissolved in dry acetonitrile (1.7 mL) and refluxed with thiourea (13.7 mg, 180 μmol) under N₂ for 4 hours. The reaction was monitored by TLC (heptane:EtOAc, 1:1) and when complete, the mixture was cooled.

### EXAMPLE 10

### Synthesis of bis-(2,4,6-tri-O-acetyl-3-azido-3-deoxy-b-D-galactopyranosyl)-sulfane (Structure 10 in Scheme 1)

The sensitive bromide compound 8 (77.0 mg, 196 μmol) and Et₃N (60 μl, 430 μmol) was added to the last mixture (compound 9). The reaction was monitored by TLC (heptane:EtOAc, 1:1). When the reaction was complete, the mixture was concentrated under reduced pressure without heating. The residue was purified by flash chromatography (Eluent: heptane:EtOAc, 1:1). ¹H NMR in CDCl₃ δ 5.50 (dd, 2H, H-4,), 5.23 (t, 2H, H-2, H-2′), 4.83 (d, 2H, H-1, H-1′), 4.15 (dd, 4H, H-6, H-6, H-6′, H-6′), 3.89 (dt, 2H, H-5, H-5′), 3.70 (dd, 2H, H-3, H-3′), 2.19 (s, 6H, 2OAc), 2.15 (s, 6H, 2OAc), 2.18 (s, 6H, 2OAc).

### EXAMPLE 11

### Synthesis of bis-(3-azido-3-deoxy-β-D-galactopyranosyl)-sulfane (Structure 11 in Scheme 1)

Compound 10 (160 mg, 0.00024 mol) was dissolved in dry MeOH (2.6 mL) and dry CH₂Cl₂ (1.6 mL), and NaOMe (1M, 24 μL, 24 μmol) was added. The reaction was monitored by TLC (heptane:EtOAc 1:1 and D:M 5:1). When the reaction was complete, the mixture was neutralized with Duolite C436 until pH 7, and was filtered and washed with MeOH. The filtered solution was concentrated under reduced pressure. The residue was purified by flash chromatography (Eluent: CH₂Cl₂:MeOH, 5:1) to give pure compound 11 (74.1 mg, 75%). 1H NMR in CDCl₃ δ 4.72 (d, 2H, J=9.7 Hz, H-1, H-1′), 3.95 (br s, 2H, H-4, H-4′), 3.84 (t, 2H, J=9.8 Hz, H-2, H-2′), 3.74 (dd, 2H, J=11.47 Hz, 7.23 Hz, H-6, H-6′), 3.64 (dd, 2H, J=11.48 Hz, 4.72 Hz, H-6, H-6′), 3.60-3.55 (ddd, 2H, 7.15 Hz, 4.67 Hz, 0.93 Hz, H-5, H-5′), 3.36 (dd, 2H, J=10 Hz, 3.05 Hz, H-3, H-3′).

### EXAMPLE 12

### Synthesis of bis-β-deoxy-{3-[4-(3-fluorophenyl)-1H-1,2,3-triazol-1-yl]-β-D-galactopyranosyl} sulfane (TD139)

TD139 was synthesized at ambient temperature by Cu(I)-catalyzed cycloaddition between bis-(3-azido-3-deoxy-β-D-galactopyranosyl)-sulfane (compound 11) and 3-fluorophenylacetylene (3 eq.) with Cu(I) (0.2 eq), triethylamine (2 eq.) in N,N-dimethylformamide (DMF, 100 mL/mmol sulfane). The reaction was monitored with TLC until complete, and concentrated and purified first by flash chromatography (Eluent: CH₂Cl₂:MeOH, 8:1), then by preparative HPLC to yield TD139 in 76% yield as a white amorphous solid. ¹H-NMR (CD₃OD, 400 MHz) d 8.59 (s, 2H, triazole-H), 7.63 (br d, 2H, 7.6 Hz, Ar—H, 7.57 (br d, 2H, 8.4 Hz, Ar—H, 7.41 (dt, 2H, 6,0 and 8.0 Hz, Ar—H, 7.05 (br dt, 2H, 2.4 and 6.4 Hz, Ar—H, 4.93 (dd, 2H, 2,4 and 10.4 Hz, H3), 4.92 (d, 2H, 10.4 Hz, H1), 4.84 (2H, 10.4 Hz, H2), 4.18 (d, 2H, 2.4 Hz, H4), 3.92 (dd, 2H, 4.2 and 7.6 Hz, H5), 3.84 (dd, 2H, 7.6 and 11.4 Hz, H6), 3.73 (dd, 2H, 4.2 and 11.4 Hz, H6); FAB-HRMS m/z calcd for C₂₈H₃₀F₂N₆NaO₈S (M+Na⁺), 671.1712; found, 671.1705.

The structure of compound TD139 is shown below:

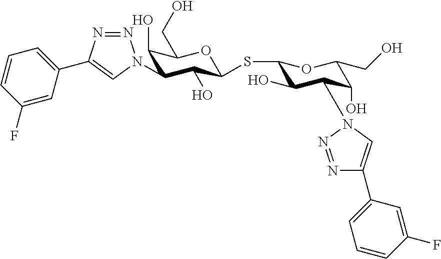

### EXAMPLE 13

### Materials and Methods

### Animals

For primary microglial cultures, galectin-3 null mutant mice (Colnot et al., 1998a) with pure (C57BL/6 background) were obtained from Dr. K. Sävman at Gothenburg University. For intracerebral injections, we purchased C57B1/6J 3-month-old female mice from Charles River Laboratories and housed them under a 12 h light/12 h dark cycle with access to food and water ad libitum at BMC animal facilities in Lund. All procedures were carried in accordance with the international guidelines and were approved by the Malmo-Lund Ethical Committee for Animal Research (M250-11).

### Genotyping

The genotype of gal3−/− and gal3+/+ mice was determined by an integrated extraction and amplification kit (Extract-N-Amp™. Sigma-Aldrich). The PCR consisted of 94° C. for 5 min, then 40 cycles with denaturation at 94° C. for 45 sec, annealing at 55° C. for 30 sec, and elongation at 72° C. for 1.5 min. The primers (CyberGene) used were as follows: galectin-3 common 5-CAC GAA CGT CTT TTG CTC TCT GG-3′ (SEQ ID NO:1)), gal3−/− 5-GCT TTT CTG GAT TCA TCG ACT GTG G-3′ (SEQ ID NO:2) (single band of 384 bp),and gal3+/+ 5-TGA AAT ACT TAC CGA AAA GCT GTC TGC-3 (SEQ ID NO:3) (single band of 300 bp) (Doverhag et al., 2008; Svedin et al., 2007). We separated the PCR products by gel electrophoresis (agarose, labelled with ethidium bromide) and visualized in a CCD camera (SONY, Tokyo, Japan).

### Cell Culture and Treatment

Murine (microglial cell line (BV2 cells)) was culture in Dulbecco's modified Eagle's medium (DMEM) containing 10% Fetal Bovine Serum (Invitrogen) with 100 U/ml Penicillin and 100 U/ml Streptomycin (Invitrogen) in 5% CO₂ atmosphere at 37° C. One day before BV2 were seeded at a concentration of 2×10⁵ cells/well in 24 wells plate (Nunc). The BV2 cells were treated with α-synuclein monomers and aggregates at different concentrations: 5 μM, 10 μM and 20 μM. The cells were also treated with LPS (Sigma-Aldrich) at 1 μg/ml. All the treatments were conducted for 12 h.

### Primary Cultures

Primary microglia culture from wild-type mice (C57 b16) and galectin-3 KO pups mice were prepared from postnatal 1-3 days and cultured as previously described (Deierborg, 2013). Briefly, cerebral cortex from mice 30 mice were dissociated in Ice Cold Hank's Balance Salt Solution without bivalent ions (HBSS) (Invitrogen), with Trypin (0.1%) (Invitrogen) and DNase (0.05%) (Sigma-Aldrich). The cells were plate in 75 cm² flask with 10 ml/flask of Dulbecco's modified Eagle's medium (DMEM)(Invitrogen) containing 10% Fetal Bovine Serum (Invitrogen) with 100 U/ml Penicillin and 100 U/ml Streptomycin (Invitrogen) in 5% CO₂ atmosphere at 37° C. After 10 days the cells can be harvested in the medium by smacking the flask 10-20 times and plate in 96 wells plate at the density of 2×10⁵ cells/well. The primary cultures were treated with α-synuclein aggregates at different concentrations: 50 nM, 200 nM, 1 μM, 5 μM and 20 μM.

### α-Synuclein Aggregates Preparation

α-synuclein monomers and α-synuclein aggregates (Protein preparations of α-synuclein monomers and α-synuclein aggregates. We analysed our α-synuclein preparations using Transmission Electron Micrograph (TEM) and western blot. Images from TEM showed signals what is expected to be small molecules in the preparation of monomers and larger molecule arrangements in our aggregated preparations, suggested monomeric and oligomeric/fibril proteins structures, respectively. Western Blot analysis confirmed monomeric protein in our monomer protein preparations that was around 15 kDa. In our aggregate protein preparation we found oligomers, that was around 30-75 kDa, monomers and a small fraction of fibrils (>250 kDa) that we used to activate the microglial cells. Monomers were used at a concentration of 70 μM and aggregates were used at 40 μM. To prepare the aggregates we used an orbital shaker at 250 rpm, shaking the monomers for 5 days at 37° C. in PBS. After 5 days incubation the proteins aggregates were sonicated using a Branson Sonifier 250 with the following conditions: 3/9 output and 30/100 Duty Cycle. We tested the composition of our aggregates and monomers using Western Blot and transmission electron microscope (TEM). We use the same concentration for monomers and aggregates, 40 μM, and applied a transmission electron microscope from Technai Spirit, Field Emission instrument (FEI, Einhofen, Holland) with a biotwin lens at 100 kV accelerating voltage. We performed negative stain of monomeric and sonicated aggregated forms of α-synuclein by using 2% uranyl acetate in water.

### Galectin-3 Inhibitor

We used a small inhibitory molecule for galectin-3 activity, bis-{3-deoxy-3-[4-(3-fluorophenyl)-1H-1,2,3-triazol-1-yl]-β-D-galactopyranosyl} sulfane (Mackinnon et al., 2012, Volarevic et al., 2012, Saksida et al., 2013). This inhibitor was used as pre-treatment for 30 min throughout the study, except in the phagocytic assay when we used 12 h incubation time. To test the inhibitor ability alter α-synuclein-induced inflammation, we tested different concentration, from 5 μM to 100 μM. After 30 min pre-treatment with the inhibitor, we add the treatments of α-synuclein monomers or aggregates of at different concentration for 12 h. The inhibitor is dilute in DMSO (40%) and distilled water (60%) in a stock concentration of 4 mM.

### Transfection Conditions

Transfection of BV2 cells was carried out using Lipofectamine 2000 (Invitrogen) following the manufacturer's recommendation. Non-targeting control and galectin-3 siRNAs were obtained from Dharmacon. (SMART pool) siRNA sequence used: siLGal3S3(1) J-041097-09 GAGAGAUACCCAUCGCUUU (SEQ ID NO:4), siLGal3S3(2) J-041097-10 ACUUCAAGGUUGCGGUCAA (SEQ ID NO:5), siLGal3S3(3) J-041097-11 ACAGUGAAACCCAACGCAA (SEQ ID NO:6), siLGal3S3(4) J-041097-12 GGAUGAAGAACCUCCGGGA (SEQ ID NO:7).

### Western Blot

Western blot was performed for iNOS and galectin-3 protein levels for BV2 microglial cell or for primary microglial cells. Briefly, 5 or 10 μg of proteins of sample were loaded on 4-20% Mini-Protean TGX Precast Gels (Bio-Rad) and the transferred to Nitrocellulose membrane (Bio-Rad) using Trans-Blot Turbo System (Bio-Rad). We blocked the membrane during 1 h at room temperature with 10% Casein (Sigma-Aldrich) diluted in PBS (tablets, Sigma-Aldrich). After blocking we incubate with the primary appropriate antibody at 4° C. overnight. We clean the membranes with PBS-Tween 20 (0.1%) and then incubate during 2 h at room temperature with the appropriate peroxidase secondary antibody (Vector Labs). Each blot was developed using Clarity Western ECL Substrate (Biorad). The protein level, measured by Bradford assay (Thermo Scientific), was normalized correcting the protein level with β-Actin. The results were normalized to the highest concentration used (20 μM) in each experiment in order to normalize inflammatory response. We also used Western Blot to estimate the composition of the α-synuclein aggregates that we generated from monomeric α-synuclein.

### Antibodies

We used the following primary antibodies: Anti-rabbit iNOS primary Antibody (1:5000, Santa Cruz), Anti-rat Galectin-3 Antibody (1:3000, M38 clone from Hakon Leffler's lab), Anti-mouse Actin 1:8000 (Sigma-Aldrich), Anti-human Synuclein antibody (Life Technology) and Anti-rabbit IBA-1 (1:500, WAKO).

### Cytokines Analysis

We measured the cytokine levels in conditioned medium from BV2 and Primary microglial cells after 12 h treatment with the MSD Th1/Th2 ultrasensitive cytokine plate (Meso Scale Discovery, USA analyzing the following cytokines IFN-γ, IL-1β, IL-2, IL-4, IL-5, KC/GRO/CINC, IL-10, IL-12 (total), and TFN-α. This electrochemiluminescence-coupled immunosorbent assay was analysed using a SECTOR Imager 6000 (Meso Scale Discovery) according to the manufacturer's recommendations. The conditioned medium was snap freezed on dry ice and kept in −80° C. freezer before analysis. We used 25 μl of sample in each case to measure the levels of different cytokines. The cytokines analysed were: IFN-γ, IL-1β, IL-2, IL-4, IL-5, IL-8, IL-10, IL-12 and TNF-α.

### Viability Assay

To test if the α-synuclein, the galectin-3 inhibitor, or both, are toxic for BV2 cells in different conditions, we used an in vitro viability assay kit based on a XTT (Sigma-Aldrich) reduction spectrophotometric method to estimate the mitochondrial activity in living cells. The assay was performed following manufacturer's protocol provided in the kit. The absorbance was measured by spectrophotometrically using a plate reader.

### Phagocytic Assay

We measure microglial phagocytosis using a phagocytosis assay kit (Cayman Chem, USA) according to the protocol provided by the manufacturer's. We plated 5×10⁴ cells/well in 96 well plates for 12 h before treating the cells with α-synuclein 20 μM for additional 12 h. Thereafter IgG-FITC beads we added with or without galectin-3 inhibitor for 12 h. The phagocytic ability was analyzed by a fluorescent plate reader (FluoStar Optima, BMG, LabTech, Sweden).

### Statistical Analysis

The differences between experimental groups were evaluated (unless otherwise stated) with one-way ANOVA with Tukey's test pos hoc, two-way ANOVA Dunnett's test pos hoc or t-test as indicated in the figure legends. P<0.05 was considered as statistically significant. For the graphs we used PRISM 6. Logaritmized data was used in the analysis of cytokines from the BV2 cells due to large intragroup variations. Normally distributed data were expressed and represented as mean±S.E.M

### Results

### α-Synuclein Promotes Microglial Activation

Different forms of α-synuclein are known to activate microglial cells (Codolo et al., 2013, Kim et al., 2013). The amount and quality of α-synuclein protein employed in our monomeric and aggregated forms were verified by western blot and electron microscopy, showing that our α-synuclein aggregates primarily contained oligomers.

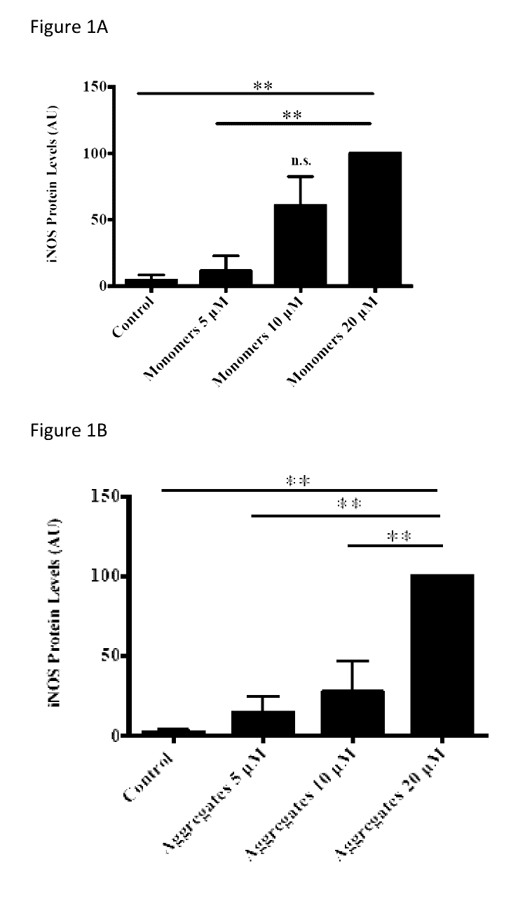

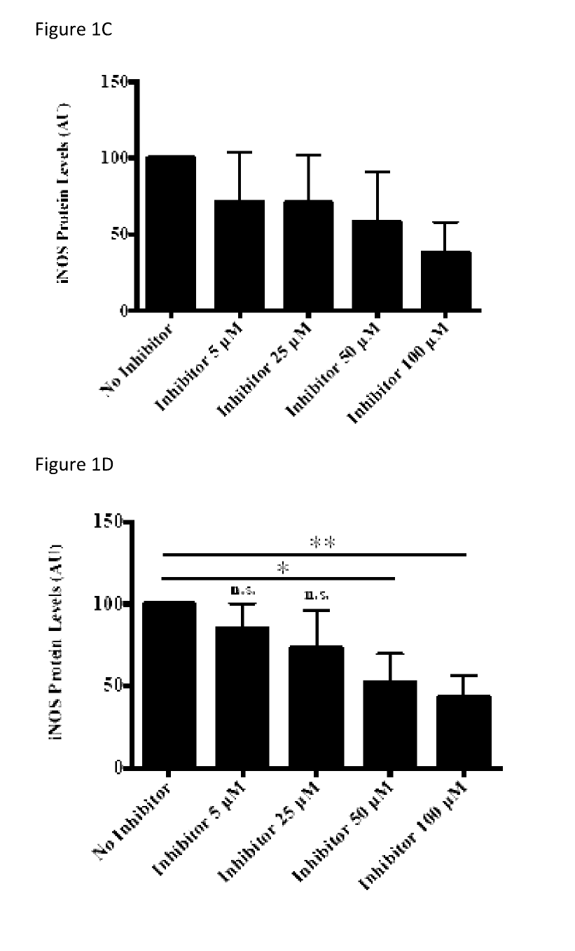

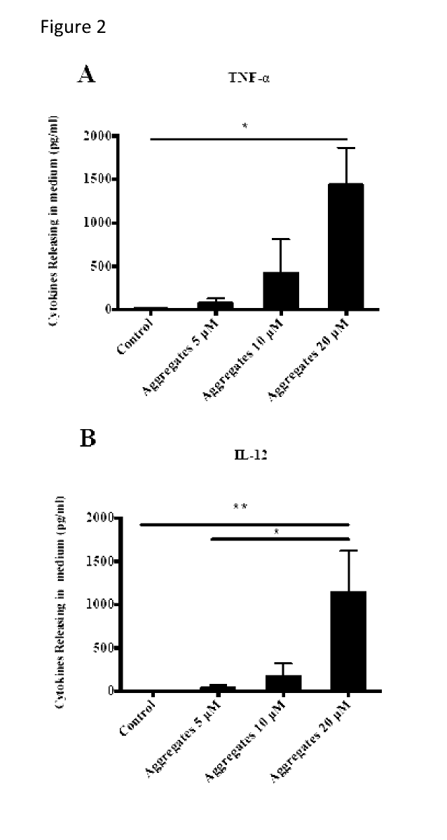

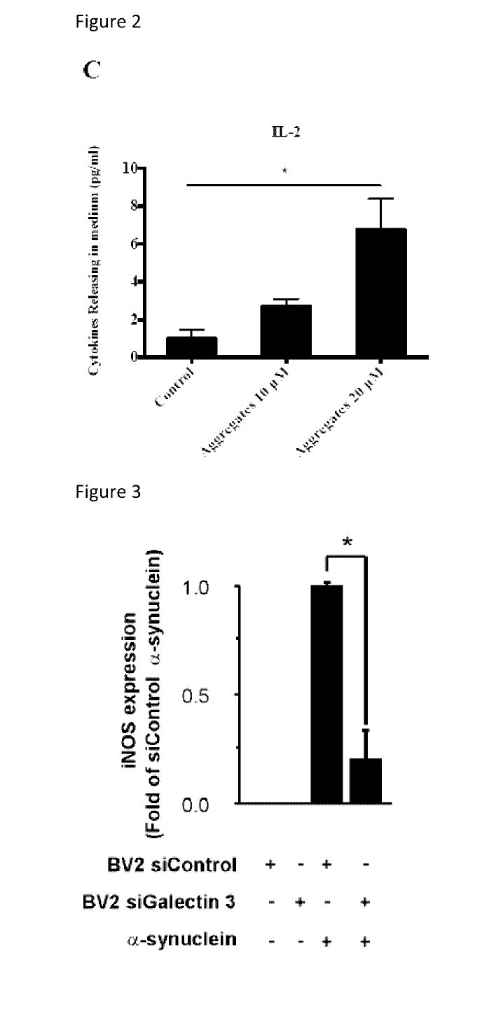

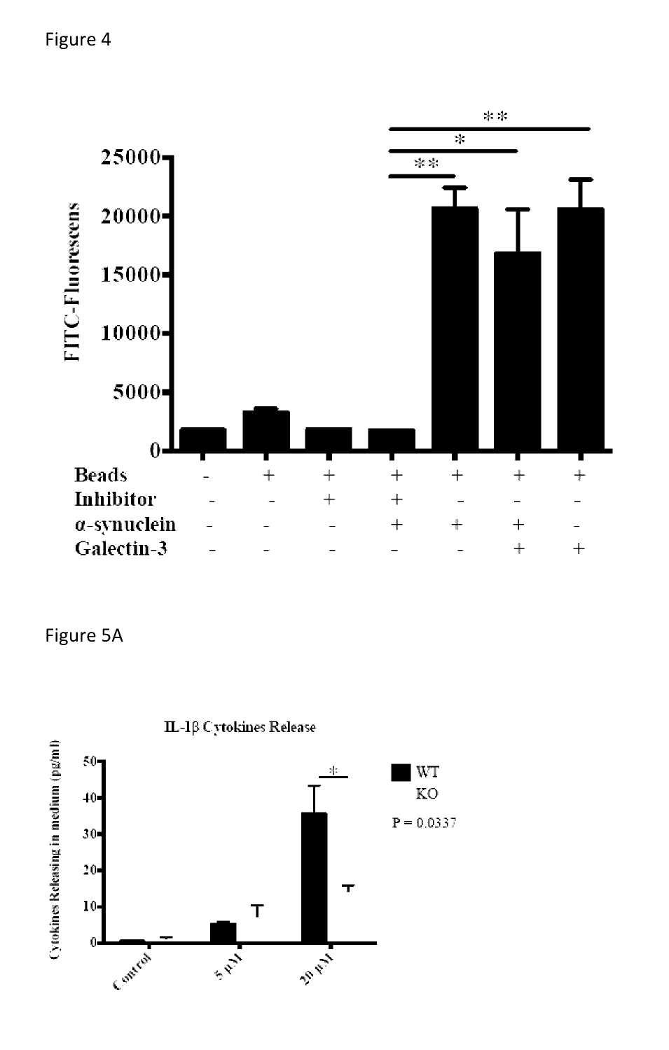

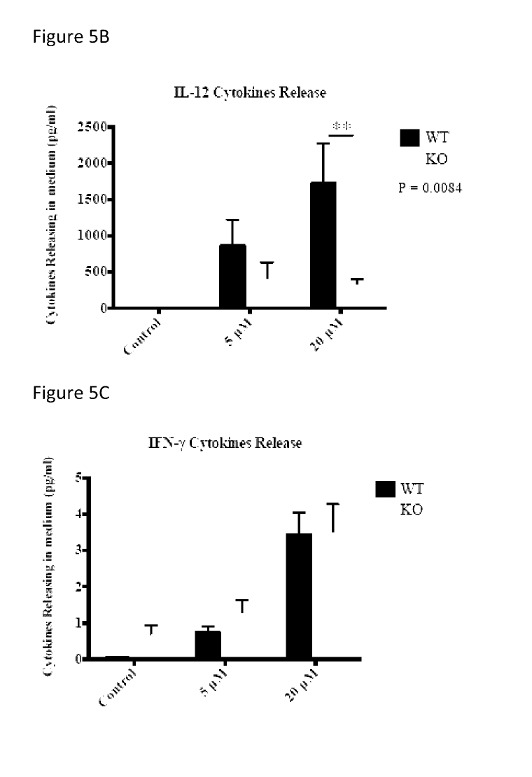

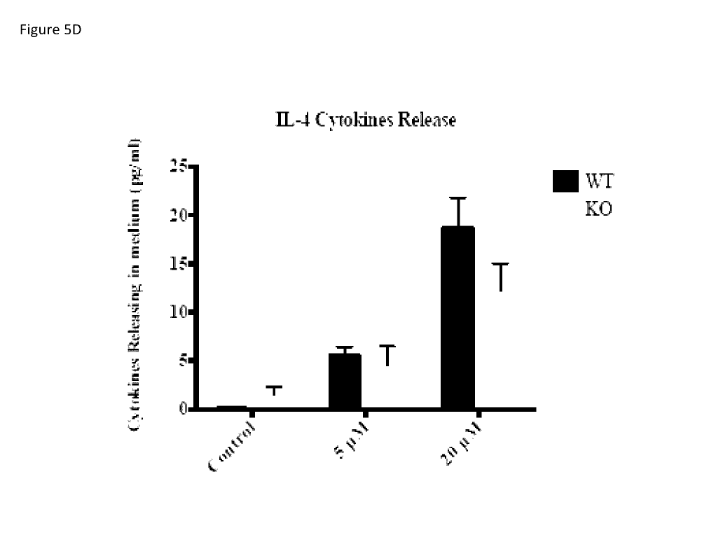

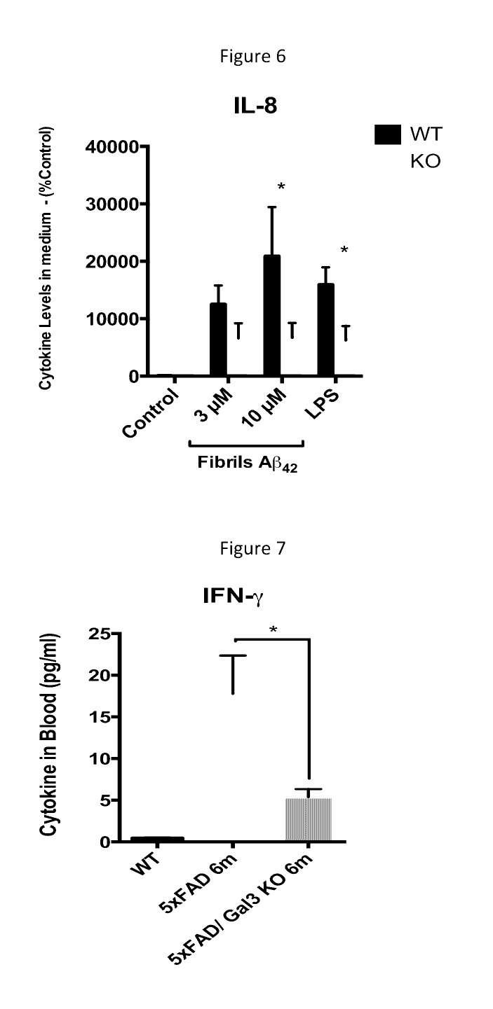

We first set out to study the inflammatory response of monomeric and aggregate forms of α-synuclein at different concentrations (5 μM, 10 μM and 20 μM). We used a 12 h incubation time based on the temporal iNOS response following LPS treatment (4, 8, 12 and 24 h), where we found a peak in the iNOS expression at 12 h (data not shown), similar to what has been reported by others (Henn et al., 2009). Using different concentrations (5 μM, 10 μM and 20 μM), we found a significant concentration-dependent upregulation of iNOS expression following both monomeric α-synuclein and aggregates of α-synuclein (FIG. 1A and FIG. 1B, respectively). However, at the maximum concentration, 20 μM, α-synuclein aggregates generating a 3-fold higher iNOS protein expression compared to monomers (P<0.05, n=3). Western blot analysis showing iNOS and β-actin protein levels. Taken together this data demonstrate that our α-synuclein preparations can successfully induce microglial activation.

### EXAMPLE 14

### Inhibition of Galectin-3 Prevents iNOS Expression in Microglial Cells

Next, we wanted to assess the role of galectin-3 inhibition in α-synuclein-induced activation of microglial cells. To this end, microglial cells were pretreated with the galectin-3 inhibitor at different concentrations (5, 25, 50 and 100 μM), for 30 min, then the cells were washed to remove the inhibitor to further treat them with α-synuclein monomers or aggregates (20 μM) for 12 h whereafter the iNOS protein expression levels were analyzed. Strikingly, we significantly inhibited α-synuclein-induced microglial activation in terms of iNOS expression after chemical inhibition of galectin-3 in a concentation-dependent manner in response to α-synuclein aggregates (FIG. 1D), but not to monomers (FIG. 1C). Indeed, more than 50% down regulation of iNOS expression was found at 50 and 100 μM inhibitor treatment.

### Galectin-3 Inhibition Do Not Impair Cell Viability

To test if the inhibitor of galectin-3 and/or α-synuclein aggregates affects cell survival, we assessed their effect using an XTT viability assay that demonstrated that galectin-3 and/or α-synuclein aggregates does not negatively affect cell viability alone or in combination with α-synuclein aggregates (data not shown).

Proinflamatory Cytokine Levels Increase after α-Synuclein Treatment

To determine the cytokine production levels of BV2 microglial cell after α-synuclein treatment, we measured cytokines in the culture medium of cells treated with α-synuclein aggregates using electrochemiluminescence ELISA. As shown in FIG. 2, there was a significant up-regulation in TNF-α, IL-12 and IL-2 secretion after 12 h incubation with α-synuclein aggregates. Noteworthy, is the fact that cytokine up-regulation occurs in a concentration dependent manner with 20 μM of protein aggregates inducing the highest cytokine secretion. Taken together, microglial activation induced by α-synuclein aggregates promotes a pro-inflammatory cascade similar to that observed in PD (Zindler and Zipp, 2010, Blandini, 2013).

Galectin-3 Knockdown in BV2 Microglial Cells Induce a Down-Regulation in iNOS Expression.

To further test the role of galectin-3 in microglial activation, we decided to knockdown galectin-3 expression in BV2 cells using small interferring RNA (siRNA). Galectin-3 and siRNA negative control knocked-down cells were treated with α-synuclein aggregates (20 μM) for 12 h and their iNOS expression levels were compared using western blot analysis. Surprisingly, we observed an 80% reduction in iNOS expression levels (P<0.05, n=3) (FIG. 3). Western blot analysis showing iNOS and β-actin protein levels. Taken together our results showed inhibition of galectin-3 using siRNA reduces α-synuclein induced microglial activation and significantly lowered iNOS protein expression.

### Pharmacological Intervention of Galectin-3 Reduces the Microglial Phagocytic Ability

Galectin-3 is known to be able to work as an opsonin and facilitate phagocytosis (Sano et al., 2003, Karlsson et al., 2009).

To test the implication of galectin-3 on phagocytic ability of microglial cells in our model of α-synuclein induced activation, we pre-treated the cells with 100 μM of galectin-3 inhibitor for 30 min and subsequently treated them with α-synuclein aggregates or combining both for 12 h. Microglial cells activated with α-synuclein show high phagocytic activity (FIG. 4). We couldn't find any difference in phagocytic ability using the inhibitor as a pre-treatment (data not shown). However, as we show in the FIG. 4, combining the inhibitor and α-synuclein for 12 h the phagocytic ability was robustly reduced to control levels upon galectin-3 inhibition by (**P<0.005). Interestingly, adding the galectin-3 protein increases the phagocytic ability to a similar extent as α-synuclein aggregates. We did not detect any synergic effect using galectin-3 and α-synuclein aggregates together. These results suggest that induction of phagocytosis is an important aspect of microglial activation by α-synuclein aggregates and that galectin-3 is an important component of this α-synuclein induced increase in phagocytosis.

Galectin-3 KO Microglial Down-Regulate iNOS Expression Following α-Synuclein Induced Activation

To further validate our results from our murine microglial cell line (BV2 cells), the role of galectin-3 on the inflammatory response was tested in primary microglial cells that we isolated from wild type and galectin-3 KO mice. We found a clear iNOS up-regulation following 12 h of α-synuclein treatment (aggregates, 20 μM) in the wild type but completely abrogated in galectin-3 KO microglia. Primary microglial culture from wild type mice show robust iNOS expression following exposure of 20 μM α-synuclein aggregates, or LPS (100 ng/ml), for 12 h. Lower concentrations of α-synuclein aggregates, 5 μM and below, failed to induce iNOS expression in wild-type microglia. Primary microglia from galectin-3 knockout mice completely lack iNOS upregulation following exposure of 20 μM α-synuclein aggregates for 12 h. Western blot analysis showed β-actin protein levels in all conditions and thereby verifying proper protein samples (n=5). These data clearly suggest that iNOS upregulation is dependent on galectin-3 (n=5).

Cytokine Levels in Primary Microglial Cells are Up-Regulated after α-Synuclein Activation

To examine the cytokine levels in primary microglial cells, we analyzed the conditioned medium after cells were treated with α-synuclein aggregates. In line with our BV2 cytokine data, we found a robust upregulation of pro-inflammatory cytokines, i.e. and IL-1β (FIG. 5A) IL-12 (FIG. 5B) and IFN-gamma (FIG. 5C) as well as for the anti-inflammatory cytokine IL-4 (FIG. 5D) after 12 h incubation with α-synuclein aggregates in wild type microglia. Importantly, microglia from knock-out mice demonstrated a significant reduction in cytokine release of IL-1β (55% reduction, P<0.05, n=5) and IL-12 (75% reduction, P<0.01, n=5) compared to wild type microglia following challenge by α-synuclein aggregate (20 μM, FIG. 5). We did not find any difference in the concentration levels of the pro-inflammatory cytokine IFN-γ or the anti-inflammatory cytokine IL-4, suggesting that blocking the function of galectin-3 inhibit certain inflammatory pathways. Taken together, our results indicate that galectin-3 is involved in the pro-inflammatory activation of certain inflammatory pathways involving the cytokines IL-1β and IL-12.

### EXAMPLE 15

To examine the potential inflammatory effect of the Alzheimer-associated protein amyloid-beta (Aβ) we challenged microglial cells with a fibrillar recombinant form of amyloid-beta, Aβ42 (the most toxic form of Aβ). The cytokine levels in primary microglial cells from galectin-3 KO were analyzed using the conditioned medium from the cells treated with amyloid-beta fibrils (human recombinant protein, Aβ42). Cytokines were measured using a high sensitivity system based on electrochemiluminescence ELISA as mentioned above (Mesoscale, MSD, US). We measured the levels of secreted cytokines in the culture medium from microglial cells challenged with Aβ42 after 12 h incubation. Importantly, microglia from galectin-3 knock-out mice demonstrated a significant reduction in cytokine release of IL-8 (see FIG. 6, 65% reduction, P<0.05, n=5) compared to wild-type microglia (n=5) challenged by Aβ42 fibrils at 10 μM Aβ42 or with LPS (1 μg/μl). This data show that galectin-3 is involved in the pro-inflammatory activation of microglia and that lack of galectin-3 robustly decrease the activation and secretion of the inflammatory cytokine IL-8 after exposure to the Alzheimer protein amyloid-beta (Aβ42). Galectin-3 stands as a key regulator of microglial phenotype, being important for shifting to an anti-inflammatory phenotype.

### EXAMPLE 16

A commonly used mouse model of AD is the 5xFAD. This mouse model has 5 different mutations related to Alzheimer's disease (PMID: 25213090). The main reason to use this model is the broad spectrum of neuropathological features related to Alzheimer Disease clearly present over the lifespan of the mice, including: amyloid-beta deposition, inflammation, glial activation, neurofibrillary tangles formation and Tau phosphorylation. This mouse model, in contrast to others, leads us to evaluate the impact of the galectin-3 modulation in almost all the main features that can be present in a human brain affected by Alzheimer disease. The neuropathological phenotype is already present after 3-4 months of age and shows a stable progression over the lifespan of the affected mice. Thereby, to study the effect of galectin-3 we have crossbreed the 5xFAD mice with galectin-3 knockout mice (Gal3KO). At the age of 6 months, we found a profound 70% down-regulation of IFN-gamma in the blood of Alzheimer mice (5XFAD) that lack galectin-3 (Gal3KO), i.e. 5xFAD/Gal3KO (n=6) compared to the normal Alzheimer mice that had galectin-3 present 5xFAD (n=5) (see FIG. 7). Cytokines were measured with electrochemiluminescence ELISA as mentioned above (Mesoscale, MSD, US). This data suggests that the pharmarcological or the genetic intervention on galectin-3 robustly down-regulate the inflammatory response. In view of the ongoing inflammation/microglial activity in the brain of Alzheimer's disease patients, we believe that lowering IFN-gamma (a key pro-inflammatory mediator for microglia) can be beneficial. Thereby, to block galectin-3 can have an effect directly on the brain, which is the most important of our finding, but can also modify the inflammatory response at systemic level being beneficial for reducing the microglial pro-inflammatory activity in the brain.

We claim:

1. A method for treatment or prevention of α-synucleinopathies comprising administering to a subject in need thereof a composition comprising a molecule for inhibition of galectin-3 activity in a mammalian brain in an amount effective for the treatment of prevention of α-synucleinopathies.

2. The method according to claim 1, wherein the molecule is selected from at least one of: a drug, a polymer, a protein, a peptide, a carbohydrate, a low molecular weight compound, an oligonucleotide, a polynucleotide, and a genetic material such as DNA or RNA.

3. The method according to claim 1, wherein the composition is effective in a method to treat or prevent a disease or a condition associated with α-synucleinpathies with inflammatory features.

4. The method according to claim 1, wherein the molecule is selected from a low molecular weight compound comprising a carbohydrate selected from a glycopyranose, a thiodigalactoside, a C3-[1,2,3]-triazol-1-yl-D-galactose, and a C3-[1,2,3]-triazol-1-yl-1-thio-D-galactose.

5. The method according to claim 1 wherein the molecule is a low molecular weight compound having a weight below 1000 Da.

6. The method according to claim 1, wherein the mammalian brain is a human brain.

7. The method according to claim 3, wherein the disease or condition is selected from a neurodegenerative disease or condition, such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy.

8. The method according to claim 7 wherein the disease or condition is selected from Parkinson's disease and Alzheimer's disease.

9. The method according to claim 1, wherein the molecule is a beta-galactoside, which is derivatized or functionalized.

10. The method according to claim 1, wherein the molecule has the following general formula: 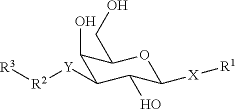
wherein the configuration of the pyranose ring is D-galacto;
X is selected from the group consisting of O, S, NH, CH₂, and NR⁴, or is a bond;
Y is selected from the group consisting of NH, CH₂, and NR⁴, or is a bond;
R¹ is selected from the group consisting of: a saccharide; hydrogen, an alkyl group, an alkenyl group, an aryl group, a heteroaryl group, and a heterocycle;
R² is selected from the group consisting of CO, SO₂, SO, PO, and PO₂;
R³ is selected from the group consisting of: an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, and an alkyl group of at least 4 carbon atoms substituted with a halogen; a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxy group, a phenyl group substituted with a carbonyl group and a phenyl group substituted with a substituted carbonyl group; and a phenyl amino group;
R⁴ is selected from the group consisting of hydrogen, an alkyl group, an alkenyl group, an aryl group, a heteroaryl group, and a heterocycle.

11. The method according to claim 1, wherein the molecule has the general formula: 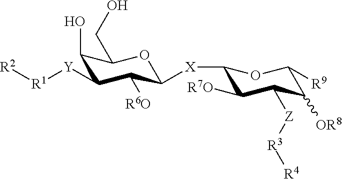
wherein the configuration of one of the pyranose rings is β-D-galacto;
X is selected from the group consisting of O, S, SO, SO₂, NH, CH₂, and NR⁵,
Y is selected from the group consisting of O, S, NH, CH₂, and NR^{S}, or is a bond;
Z is selected from the group consisting of O, S, NH, CH₂, and NR^{S}, or is a bond;
R¹ and R³ are independently selected from the group consisting of CO, SO₂, SO, PO₂, PO, and CH₂ or is a bond;
R² and R⁴ are independently selected from the group consisting of: an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamnino group, a phenyl group substituted with at least one hydroxy group, a phenyl group substituted with at least one carbonyl group and a phenyl group substituted with at least one substituted carbonyl group; or a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamnino group, a naphthyl group substituted with at least one hydroxy group, a naphthyl group substituted with at least one carbonyl group and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group; R⁶ and R⁸ are independently selected from the group consisting of a hydrogen, an acyl group, an alkyl group, a benzyl group, and a saccharide; R⁷ is selected from the group consisting of a hydrogen, an acyl group, an alkyl group, and a benzyl group; R⁹ is selected from the group consisting of a hydrogen, a methyl group, hydroxymethyl group, an acyloxymethyl group, an alkoxymethyl group, and a benzyloxymethyl group.

12. The method according to claim 1, wherein the molecule has the general formula: 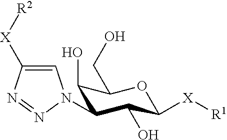
wherein the configuration of the pyranose ring is D-galacto;
X is selected from the group consisting of O, S, NH, CH₂, and NR⁴, or is a bond;
Y is selected from the group consisting of CH₂, CO, SO₂, SO, PO₂ and PO, phenyl, or is a bond;
R₁ is selected from the group consisting of: a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives thereof; and an amino group, a substituted amino group, an imino group, or a substituted imino group; and,
R² is selected from the group consisting of; hydrogen, an amino group, a substituted amino group, an alkyl group, a substituted alkyl group, an alkenyl group, a substituted alkenyl group, an alkynyl group, a substituted alkynyl group, an alkoxy group, a substituted alkoxy group, an alkylamino group, a substituted alkylamino group, an arylamino group, a substituted arylamino group, an aryloxy group, a substituted aryloxy group, an aryl group, a substituted aryl group, a heteroaryl group, a substituted heteroaryl group, and a heterocycle, a substituted heterocycle.

13. The method according of claim 1, wherein the molecule has the general formula shown below: 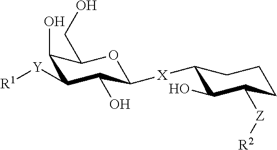
wherein the configuration of the pyranose ring is D-galacto;
X is selected from the group consisting of O, S, and SO;
Y and Z are independently selected from: CONH or a 1H-1,2,3-triazole ring; R¹ and R² are independently selected from the group consisting of: an alkyl group of at least 4 carbons, an alkenyl group of at least 4 carbons, an alkynyl group of at least 4 carbons; a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, and a carbamoyl group substituted with an substituted aryl group; a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group; a phenyl group substituted with at least one trifluoromethoxy group, a phenyl group substituted with at least one sulfo group, a phenyl group substituted with at least one hydroxy group, a phenyl group substituted with at least one carbonyl group, and a phenyl group substituted with at least one substituted carbonyl group; a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, and a heteroaryl group substituted with at least one substituted carbonyl group; and a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group.

14. The method according to claim 1, wherein the molecule has the general formula (13) 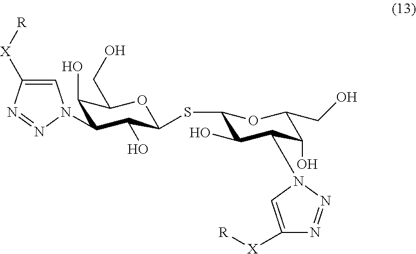
wherein the configuration of at least one of the pyranose rings is D-galacto; X is a bond; R is a phenyl group, which is substituted in any position with one or more substituents selected from the group consisting of methyl, ethyl, isopropyl, tert-butyl, fluoro, chloro, bromo, and trifluoromethyl or R is a thienyl group.

15. The method according to claim 1, wherein the molecule is bis-{3-deoxy-3-[4-(3-fluorophenyl)-1H-1,2,3-triazol-1-yl]-β-D-galactopyranosyl}sulfane (TD139), optionally as the free form, such as crystalline form.


SEQUENCE LISTING (7 sequences)

SEQ ID NO: 1 - DNA, 23 bp, Artificial Sequence
  Description of Artificial Sequence Synthetic primer
cacgaacgtcttttgctctctgg

SEQ ID NO: 2 - DNA, 25 bp, Artificial Sequence
  Description of Artificial Sequence Synthetic primer
gcttttctggattcatcgactgtgg

SEQ ID NO: 3 - DNA, 27 bp, Artificial Sequence
  Description of Artificial Sequence Synthetic primer
tgaaatacttaccgaaaagctgtctgc

SEQ ID NO: 4 - RNA, 19 nt, Artificial Sequence
  Description of Artificial Sequence Synthetic oligonucleotide
gagagauacccaucgcuuu

SEQ ID NO: 5 - RNA, 19 nt, Artificial Sequence
  Description of Artificial Sequence Synthetic oligonucleotide
acuucaagguugcggucaa

SEQ ID NO: 6 - RNA, 19 nt, Artificial Sequence
  Description of Artificial Sequence Synthetic oligonucleotide
acagugaaacccaacgcaa

SEQ ID NO: 7 - RNA, 19 nt, Artificial Sequence
  Description of Artificial Sequence Synthetic oligonucleotide
ggaugaagaaccuccggga